(19) United States
(12) Patent Application Publication
(10) Pub. No.: US 2024/0190969 A1
(43) Pub. Date: Jun. 13, 2024

(54) SPECIFIC BINDING MOLECULES

(71) Applicant: IMMUNOCORE LIMITED, Abingdon, Oxfordshire (GB)

(72) Inventors: Chandramouli CHILLAKURI, Abingdon, Oxfordshire (GB); Jorge DIAS DO NASCIMENTO, Abingdon, Oxfordshire (GB); Conor HAYES, Abingdon, Oxfordshire (GB); James PARKER, Abingdon, Oxfordshire (GB); Peter JAMES, Abingdon, Oxfordshire (GB); Ronan O'DWYER, Abingdon, Oxfordshire (GB); Andrew POOLE, Abingdon, Oxfordshire (GB)

(21) Appl. No.: 17/771,447

(22) PCT Filed: Oct. 21, 2020
(86) PCT No.: PCT/EP2020/079573

§ 371 Date: Apr. 22, 2022

(30) Foreign Application Priority Data
Oct. 22, 2019 (GB) 1915282.6

(51) Int. Cl. C07K 16/28; A61K 35/17; A61K 39/00; A61P 35/00

(52) U.S. Cl. CPC C07K 16/2833; A61K 35/17; A61P 35/00; C07K 16/2809; A61K 2039/505; C07K 2317/565; C07K 2317/567; C07K 2319/74

(57) ABSTRACT

The present invention relates to specific binding molecules, such as T cell receptors (TCRs), that bind the HLA-A*02 restricted peptide KVLEYVIKV (SEQ ID NO: 1) derived from the germline cancer antigen MAGEA1. The specific binding molecules may comprise non-natural mutations within the alpha and/or beta variable domains relative to a native MAGEA1 TCR. The specific binding molecules of the invention are particularly suitable for use as novel immunotherapeutic reagents for the treatment of malignant disease.

The present invention relates to specific binding molecules, such as T cell receptors (TCRs), that bind the HLA-A*02 restricted peptide KVLEYVIKV (SEQ ID NO: 1) derived from the germline cancer antigen MAGEA1. Said specific binding molecules may comprise non-natural mutations within the alpha and/or beta variable domains relative to a native MAGEA1 TCR. The specific binding molecules of the invention are particularly suitable for use as novel immunotherapeutic reagents for the treatment of malignant disease.

### BACKGROUND TO THE INVENTION

T cell receptors (TCRs) are naturally expressed by CD4⁺ and CD8⁺ T cells. TCRs are designed to recognize short peptide antigens that are displayed on the surface of antigen presenting cells in complex with Major Histocompatibility Complex (MHC) molecules (in humans, MHC molecules are also known as Human Leukocyte Antigens, or HLA) (Davis et al., Annu Rev Immunol. 1998; 16:523-44). CD8⁺ T cells, which are also termed cytotoxic T cells, have TCRs that specifically recognize peptides bound to MHC class I molecules. CD8⁺ T cells are generally responsible for finding and mediating the destruction of diseased cells, including cancerous and virally infected cells. The affinity of cancer-specific TCRs in the natural repertoire for corresponding antigen is typically low as a result of thymic selection, meaning that cancerous cells frequently escape detection and destruction. Novel immunotherapeutic approaches aimed at promoting cancer recognition by T cells offer a highly promising strategy for the development of effective anticancer treatments.

MAGEA1 (Melanoma-associated antigen 1) is a member of the family of germline-encoded antigens known as cancer testis antigens. Cancer testis antigens are attractive targets for immunotherapeutic intervention since they typically have limited or no expression in normal adult tissues. MAGEA1 has Uniprot accession number P43355 and is also known as MAGE-1 antigen, Cancer/testis antigen 1.1, CT1.1 or antigen MZ2-E. MAGEA1 is expressed in a number of solid tumours as well as in leukaemias and lymphomas (refs). Immunotherapies that target MAGEA1 targeting therapies of the inventions may be particularly suitable for treatment cancers including, but not limited to, lung (NSCLC and SCLC), breast (including triple negative), ovarian, endometrial, oesophageal, bladder and head and neck cancers.

The peptide KVLEYVIKV (SEQ ID NO: 1) corresponds to amino acids 278-286 of the full length MAGEA1 protein and is presented on the cell surface in complex with HLA-A*02. This peptide-HLA complex provides a useful target for TCR-based immunotherapeutic intervention.

The identification of particular TCR sequences that bind to the KVLEYVIKV (SEQ ID NO: 1) HLA-A*02 complex with high affinity and high specificity is advantageous for the development of novel immunotherapies. Therapeutic TCRs may be used, for example, as soluble targeting agents for the purpose of delivering cytotoxic agents to the tumour site or activating immune effector functions against the tumour cells (Lissin, et al., “High-Affinity Monocloncal T-cell receptor (mTCR) Fusions” in Fusion Protein Technologies for Biophamaceuticals: Applications and Challenges. 2013. S. R. Schmidt, Wiley; Boulter et al., Protein Eng. 2003 September; 16(9):707-11; Liddy, et al., Nat Med. 2012 June; 18(6):980-7), or alternatively they may be used to engineer T cells for adoptive therapy.

TCRs that bind to KVLEYVIKV (SEQ ID NO: 1) in complex with HLA-A*02 have been reported previously (WO2014118236, CN106749620 WO2018104438, WO2018170338). However, these TCRs have not been engineered (mutated) so that they bind to the target antigen with increased affinity/supra-physiological affinity, relative to the natural TCR. As explained further below, supra-physiological antigen affinity is a desirable feature for a therapeutic TCR, the production of which is not straightforward, particularly when balanced with other desirable features, such as specificity.

The TCR sequences defined herein are described with reference to IMGT nomenclature which is widely known and accessible to those working in the TCR field. For example, see: LeFranc and LeFranc, (2001). “T cell Receptor Factsbook”, Academic Press; Lefranc, (2011), Cold Spring Harb Protoc 2011(6): 595-603; Lefranc, (2001), Curr Protoc Immunol Appendix 1: Appendix 10O; and Lefranc, (2003), Leukemia 17(1): 260-266. Briefly, αβ TCRs consist of two disulphide linked chains. Each chain (alpha and beta) is generally regarded as having two domains, namely a variable and a constant domain. A short joining region connects the variable and constant domains and is typically considered part of the alpha variable region. Additionally, the beta chain usually contains a short diversity region next to the joining region, which is also typically considered part of the beta variable region.

The variable domain of each chain is located N-terminally and comprises three Complementarity Determining Regions (CDRs) embedded in a framework sequence (FR). The CDRs comprise the recognition site for peptide-MHC binding. There are several genes coding for alpha chain variable (Vα) regions and several genes coding for beta chain variable (Vβ) regions, which are distinguished by their framework, CDR1 and CDR2 sequences, and by a partly defined CDR3 sequence. The Vα and Vβ genes are referred to in IMGT nomenclature by the prefix TRAV and TRBV respectively (Folch and Lefranc, (2000), Exp Clin Immunogenet 17(1): 42-54; Scaviner and Lefranc, (2000), Exp Clin Immunogenet 17(2): 83-96; LeFranc and LeFranc, (2001), “T cell Receptor Factsbook”, Academic Press). Likewise there are several joining or J genes, termed TRAJ or TRBJ, for the alpha and beta chain respectively, and for the beta chain, a diversity or D gene termed TRBD (Folch and Lefranc, (2000), Exp Clin Immunogenet 17(2): 107-114; Scaviner and Lefranc, (2000), Exp Clin Immunogenet 17(2): 97-106; LeFranc and LeFranc, (2001), “T cell Receptor Factsbook”, Academic Press). The huge diversity of T cell receptor chains results from combinatorial rearrangements between the various V, J and D genes, which include allelic variants, and junctional diversity (Arstila, et al., (1999), Science 286(5441): 958-961; Robins et al., (2009), Blood 114(19): 4099-4107.) The constant, or C, regions of TCR alpha and beta chains are referred to as TRAC and TRBC respectively (Lefranc, (2001), Curr Protoc Immunol Appendix 1: Appendix 10).

The inventors of the present application have surprisingly found novel TCRs that are able to bind to KVLEYVIKV -HLA-A*02 complex with high affinity and specificity. Certain specific binding molecules of the invention are engineered from a suitable scaffold sequence into which a number of mutations are introduced. The specific binding molecules of the invention have a particularly suitable profile for therapeutic use. In general, the identification of such TCRs is not straightforward and typically has a high attrition rate, so there is no expectation of success in identifying a suitable specific binding molecule (e.g. TCR) for a particular target.

In the first instance, the skilled person needs to identify a suitable starting, or scaffold, sequence. Typically such sequences are obtained from natural sources e.g. from antigen responding T cells extracted from donor blood. Given the rarity of cancer specific T cells in the natural repertoire, it is often necessary to screen many donors, for example 20 or more, before a responding T cell may be found. The screening process may take several weeks or months, and even where a responding T cell is found, it may be unsuitable for immunotherapeutic use. For example, the response may be too weak and/or may not be specific for the target antigen. Alternatively, it may not be possible to generate a clonal T cell population, nor expand or maintain a given T cell line to produce sufficient material to identify the correct TCR chain sequences. TCR sequences that are suitable as starting, or scaffold, sequences should have one or more of the following properties: a good affinity for the target peptide-HLA complex, for example 200 μM or stronger; a high level of target specificity, e.g. relatively weak or no binding to alternative peptide-HLA complexes; be amenable to use in display libraries, such as phage display; and be able to be refolded and purified at high yield. Given the degenerate nature of TCR recognition, it is exceptionally hard even for skilled practitioners to be able to determine whether a particular scaffold TCR sequence has a specificity profile that would make it eligible for engineering for therapeutic use (Wooldridge, et al., J Biol Chem. 2012 Jan. 6; 287(2):1168-77).

The next challenge is to engineer the TCR to have a higher affinity towards the target antigen whilst retaining desirable characteristics such as specificity and yield. TCRs, as they exist in nature, have weak affinity for target antigen (low micromolar range) compared with antibodies, and TCRs against cancer antigens typically have weaker antigen recognition than viral specific TCRs (Aleksic, et al. Eur J Immunol. 2012 December; 42(12):3174-9). This weak affinity coupled with HLA down-regulation on cancer cells means that therapeutic TCRs for cancer immunotherapy typically require engineering to increase their affinity for target antigen and thus generate a more potent response. Such affinity increases are essential for soluble TCR-based reagents. In such cases, antigen-binding affinities in the nanomolar to picomolar range, with binding half-lives of several hours, are desirable. The improved potency generated by high affinity antigen recognition at low epitope numbers is exemplified in FIGS. 1e and 1f of Liddy et al. (Liddy, et al., Nat Med. 2012 June; 18(6):980-7). The affinity maturation process, typically involves the skilled person having to engineer specific mutations and/or combinations of mutations, including but not limited to substitutions, insertions and/or deletions, on to the starting TCR sequence in order to increase the strength of antigen recognition. Methods to engineer affinity enhancing mutations on to a given TCR are known in the art, for example the use of display libraries (Li et al., Nat Biotechnol. 2005 March; 23(3):349-54; Holler et al., Proc Natl Acad Sci USA. 2000 May 9; 97(10):5387-92). However, to produce significant increases in the affinity of a given TCR against a given target, the skilled person may have to engineer combinations of mutations from a large pool of possible alternatives. The specific mutations and/or combinations of mutations that produce significant increases in affinity are not predictable and there is a high attrition rate. In many cases, it may not be possible to achieve significant increases in affinity with a given TCR starting sequence.

The affinity maturation process must also take account of the necessity of maintaining TCR antigen specificity. Increasing the affinity of a TCR for its target antigen brings a substantial risk of revealing cross reactivity with other unintended targets as a result of the inherent degeneracy of TCR antigen recognition (Wooldridge, et al., J Biol Chem. 2012 Jan. 6; 287(2):1168-77; Wilson, et al., Mol Immunol.2004 February; 40(14-15):1047-55; Zhao et al., J Immunol. 2007 Nov. 1; 179(9):5845-54). At a natural level of affinity the recognition of the cross reactive antigen may be too low to produce a response. If a cross reactive antigen is displayed on normal healthy cells, there is a strong possibility of off-target binding in vivo which may manifest in clinical toxicity. Thus, in addition to increasing antigen binding strength, the skilled person must also engineer mutations and or combinations of mutations that allow the TCR to retain a high specificity for target antigen and demonstrate a good safety profile in preclinical testing. Again, suitable mutations and/or combinations of mutations are not predictable. The attrition rate at this stage is even higher and in many cases may not be achievable at all from a given TCR starting sequence.

Despite the difficulties described above, the inventors have identified mutated TCRs with a particularly suitable affinity and specificity. Said TCRs demonstrate potent and specific killing of antigen positive cancer cells, when prepared as soluble reagents fused to a T cell redirecting moiety.

### DESCRIPTION OF THE INVENTION

In a first aspect, the present invention provides a specific binding molecule having the property of binding to KVLEYVIKV (SEQ ID NO: 1) in complex with HLA-A*02 and comprising a TCR alpha chain variable domain and/or a TCR beta chain variable domain, each of which comprises FR1-CDR1-FR2-CDR2-FR3-CDR3-FR4 where FR is a framework region and CDR is a complementarity determining region, wherein,
(a) the alpha chain CDRs have the following sequences:

CDR1-
(SEQ ID NO: 15)
SSVPPY
CDR2-
(SEQ ID NO: 16)
YTSAATLV
CDR3-
(SEQ ID NO: 17)
AARPSSSNTGKLI

optionally with one or more mutations therein,
   and/or
(b) the beta chain CDRs have the following sequences:

CDR1-
(SEQ ID NO: 18)
PRHDT
CDR2-
(SEQ ID NO: 19)
FYEKMQ
CDR3-
(SEQ ID NO: 20)
ASSFTGFDEQF

optionally with one or more mutations therein.

The invention provides specific binding molecules including CDRs and variable domains, which bind to the KVLEYVIKV-HLA complex. The specific binding molecules or binding fragments thereof include TCR variable domains, which may correspond to those from a native TCR, or more preferably the TCR variable domains may be engineered/non-natural. Native TCR variable domains may also be referred to as wild-type, natural, parental, unmutated or scaffold domains. The specific binding molecules or binding fragments can be used to produce molecules with ideal therapeutic properties such as supra-physiological affinity for target, long binding half-life, high specificity for target and good stability. The invention also includes bispecific, or bifunctional, or fusion molecules that incorporate specific binding molecules or binding fragments thereof and a T cell redirecting moiety. Such molecules can mediate a potent and specific response against antigen positive target cells by re-directing and activating T-cells. Alternatively, the specific binding molecules or binding fragments may be incorporated into engineered T cells for adoptive therapy.

In the specific binding molecule of the first aspect, the alpha chain variable domain framework regions may comprise the following framework sequences:
FR1—amino acids 1-26 of SEQ ID NO: 2
FR2—amino acids 33-49 of SEQ ID NO: 2
FR3—amino acids 58-91 of SEQ ID NO: 2
FR4—amino acids 105-115 of SEQ ID NO: 2
or respective sequences having at least 90, at least 91, at least 92, at least 93, at least 94, at least 95, at least 96, at least 97, at least 98 or at least 99% identity to said sequences, and/or the beta chain variable domain framework regions may comprise the following sequences:
   FR1—amino acids 1-26 of SEQ ID NO: 3
   FR2—amino acids 32-48 of SEQ ID NO: 3
   FR3—amino acids 55-91 of SEQ ID NO: 3
   FR4—amino acids 103-112 of SEQ ID NO: 3
or respective sequences having at least 90, at least 91, at least 92, at least 93, at least 94, at least 95, at least 96, at least 97, at least 98 or at least 99% identity to said sequences.

The alpha chain framework region may comprise an amino acid sequence corresponding to a TRAV8-4*01 chain and/or the beta chain framework region may comprise an amino acid sequence corresponding to those of a TRBV13*01 chain. The framework region may have at least 90, at least 91, at least 92, at least 93, at least 94, at least 95, at least 96, at least 97, at least 98 or at least 99% identity to a TRAV8-4*01 chain or a TRBV13*01 chain.

As used herein, the term “specific binding molecule” refers to a molecule capable of binding to a target antigen. Such molecules may adopt a number of different formats as discussed herein. One example is a TCR, another is a diabody comprising TCR CDRs, which may be in the form of TCR variable regions. Furthermore, fragments of the specific binding molecules of the invention are also envisioned. A fragment refers to a portion of the specific binding molecule that retains binding to the target antigen.

The term ‘mutations’ encompasses substitutions, insertions and deletions. Mutations to a native (also referred to as parental, natural, unmutated wild type, or scaffold) specific binding molecule may include those that increase the binding affinity (k_{D} and/or binding half life) of the specific binding molecule to KVLEYVIKV-HLA-A*02 complex.

The alpha chain CDRs and/or framework regions may contain at least one mutation relative to the native CDRs and or framework. There may be one, two or fewer, three or fewer, four or fewer or five or fewer, six or fewer, seven or fewer, eight or fewer, nine or fewer, ten or fewer, or more, mutations in the alpha chain CDRs and or framework. The alpha chain CDRs and or framework may contain substitutions and insertions. The alpha chain may contain the following CDR mutations with reference to the numbering of SEQ ID NO: 2:
Insertion of 4 amino acids (e.g. ARWG) after position 26
S27D
S28G
S52G
A53G
A54D
T55L
I56V
S97D
S98A

The beta chain CDRs and/or framework regions may contain at least one mutation relative to the native CDRs and or framework. There may be one or fewer, two or fewer, three or fewer, four or fewer, five or fewer, six or fewer, seven or fewer, eight or fewer, or more, mutations in the beta chain CDRs and or framework. The beta chain CDRs and or framework may contain substitutions and insertions.

The beta chain may contain the following CDR mutations with reference to the numbering of SEQ ID NO: 3:
Y50F
K52T
M53K
Q54F
F95V
T96W
G97D
F98W/Y

Thus, there may be any or all of the listed mutations in the alpha and beta chain CDRs, optionally in combination with other mutations. Other mutations may be in the CDRs and or in the framework regions.

The alpha chain CDR1 may comprise the following sequence:

(SEQ ID NO: 15)
SSVPPY
or
(SEQ ID NO: 21)
ARWGDGVPPY.

The alpha chain CDR2 may comprise the following sequence:

(SEQ ID NO: 16)
YTSAATLV
or
(SEQ ID NO: 22)
YTGGDLVV.

The alpha chain CDR3 may comprise the following sequence:

(SEQ ID NO: 17)
AARPSSSNTGKLI
(SEQ ID NO: 23)
AARPSDSNTGKLI
or
(SEQ ID NO: 24)
AARPSSANTGKLI.

The beta chain CDR1 may comprise the following sequence:

(SEQ ID NO: 18)
PRHDT,

The beta chain CDR2 may comprise the following sequence:

(SEQ ID NO: 19)
FYEKMQ.
(SEQ ID NO: 25)
FFETMF
or
(SEQ ID NO: 26)
FFETKF

The betachain CDR3 may comprise the following sequence:

(SEQ ID NO: 20)
ASSFTGFDEQF
(SEQ ID NO: 27)
ASSVWDWDEQF
or
(SEQ ID NO: 28)
ASSVWDYDEQF.

Preferred combinations of alpha and beta chain CDRs are shown in the table below

| Alpha | | | Beta | | |
|---|---|---|---|---|---|
| CDR1 | CDR2 | CDR3 | CDR1 | CDR2 | CDR3 |
| ARWGDGVPPY | YTGGDLVV | AARPSSSNTGKLI | PRHDT | FFETMF | ASSVWDWDEQF |
| ARWGDGVPPY | YTGGDLVV | AARPSSSNTGKLI | PRHDT | FFETKF | ASSVWDYDEQF |
| ARWGDGVPPY | YTGGDLVV | AARPSDSNTGKLI | PRHDT | FFETMF | ASSVWDWDEQF |
| ARWGDGVPPY | YTGGDLVV | AARPSDSNTGKLI | PRHDT | FFETMF | ASSVWDYDEQF |
| ARWGDGVPPY | YTGGDLVV | AARPSSANTGKLI | PRHDT | FFETMF | ASSVWDYDEQF |

A particularly preferred combination is shown in the table below:

| Alpha | | | Beta | | |
|---|---|---|---|---|---|
| CDR1 | CDR2 | CDR3 | CDR1 | CDR2 | CDR3 |
| ARWGDGVPPY | YTGGDLVV | AARPSSSNTGKLI | PRHDT | FFETKF | ASSVWDYDEQF |

Another particularly preferred combination is shown in the table below:

| Alpha | | | Beta | | |
|---|---|---|---|---|---|
| CDR1 | CDR2 | CDR3 | CDR1 | CDR2 | CDR3 |
| ARWGDGVPPY | YTGGDLVV | AARPSSSNTGKLI | PRHDT | FFETMF | ASSVWDWDEQF |

Mutation(s) within the CDRs preferably improve the binding affinity of the specific binding molecule to the KVLEYVIKV-HLA-A*02 complex, but may additionally or alternatively confer other advantages such as improved stability in an isolated form and improved specificity. Mutations at one or more positions may additionally or alternatively affect the interaction of an adjacent position with the cognate pMHC complex, for example by providing a more favourable angle for interaction. Mutations may include those that are able to reduce the amount of non-specific binding, i.e. reduce binding to alternative antigens relative to KVLEYVIKV-HLA-A*02. Mutations may include those that increase efficacy of folding and/or manufacture. Some mutations may contribute to each of these characteristics; others may contribute to affinity but not to specificity, for example, or to specificity but not to affinity etc.

Typically, at least 5, at least 10, at least 15, or more CDR mutations in total are needed to obtain specific binding molecules with pM affinity for target antigen. Specific binding molecules with pM affinity for target antigen are especially suitable for soluble therapeutics. Specific binding molecules for use in adoptive therapy applications may have lower affinity for target antigen and thus fewer CDR mutations, for example, up to 1, up to 2, up to 5, or more CDR mutations in total. In some cases, specific binding molecules may have a sufficiently high affinity for target antigen and thus no mutations are required.

Mutations may additionally, or alternatively, be made outside of the CDRs, within the framework regions; such mutations may improve binding, and/or specificity, and/or stability, and/or the yield of a purified soluble form of the specific binding molecule. For example, the N terminus of the alpha and/or beta chain may be modified to improve the efficiency of N terminal methionine cleavage during production in E. coli. Inefficient cleavage may be detrimental for a therapeutic, since it may result in a heterogeneous protein product, and or the presence of the initiation methionine may be immunogenic in humans.

Preferably, the alpha chain variable domain of the specific binding molecule of the invention may comprise respective framework amino acid sequences that have at least 90%, at least 91%, at least 92%, at least 93%, at least 94%, at least 95%, at least 96%, at least 97%, at least 98% or at least 99% identity to the framework amino acid residues 1-26, 33-49, 58-91, 105-115 of SEQ ID NO: 2. The beta chain variable domain of the specific binding molecule of the invention may comprise respective framework amino acid sequences that have at least 90%, at least 91%, at least 92%, at least 93%, at least 94%, at least 95%, at least 96%, at least 97%, at least 98% or at least 99% identity to the framework amino acid residues 1-26, 32-48, 55-91, 103-112 of SEQ ID NO: 3. Alternatively, the stated percentage identity may be over the framework sequences when considered as a whole.

The alpha chain variable domain may comprise any one of the amino acid sequences of SEQ ID NOs: 4, 5 and 6 or a sequence having at least 90% identity thereto and the beta chain variable domain may comprise any one of the amino acid sequences of SEQ ID NOs: 7, 8 and 9 or a sequence having at least 90% identity thereto.

For example, the specific binding molecule may comprise the following alpha and beta chain variable domain pairs:

| Alpha chain variable domain | Beta chain variable domain |
|---|---|
| SEQ ID No: 4 | SEQ ID No: 7 |
| SEQ ID No: 4 | SEQ ID No: 8 |
| SEQ ID No: 5 | SEQ ID No: 7 |
| SEQ ID No: 5 | SEQ ID No: 9 |
| SEQ ID No: 6 | SEQ ID No: 9 |

A preferred pairing is SEQ ID NO: 4 and SEQ ID NO: 8. Another preferred pairing is SEQ ID NO: 4 and SEQ ID NO: 7.

Within the scope of the invention are phenotypically silent variants of any specific binding molecule of the invention disclosed herein. As used herein the term “phenotypically silent variants” is understood to refer to a specific binding molecule with a TCR variable domain which incorporates one or more further amino acid changes, including substitutions, insertions and deletions, in addition to those set out above, which TCR has a similar phenotype to the corresponding TCR without said change(s). For the purposes of this application, TCR phenotype comprises binding affinity (K_{D} and/or binding half-life) and specificity. Preferably, the phenotype of a soluble specific binding molecule associated with an immune effector includes potency of immune activation and purification yield, in addition to binding affinity and specificity. A phenotypically silent variant may have a K_{D} and/or binding half-life for the KVLEYVIKV -HLA-A*02 complex within 50%, or more preferably within 30%, 25% or 20%, of the measured KD and/or binding half-life of the corresponding TCR without said change(s), when measured under identical conditions (for example at 25° C. and/or on the same SPR chip). Suitable conditions are further provided in Example 2. As is known to those skilled in the art, it may be possible to produce specific binding molecules that incorporate changes in the variable domains thereof compared to those detailed above without altering the affinity of the interaction with the KVLEYVIKV-HLA-A*02 complex, and or other functional characteristics. In particular, such silent mutations may be incorporated within parts of the sequence that are known not to be directly involved in antigen binding (e.g. the framework regions and or parts of the CDRs that do not contact the antigen). Such variants are included in the scope of this invention.

Phenotypically silent variants may contain one or more conservative substitutions and/or one or more tolerated substitutions. By tolerated substitutions it is meant those substitutions which do not fall under the definition of conservative as provided below but are nonetheless phenotypically silent. The skilled person is aware that various amino acids have similar properties and thus are ‘conservative’. One or more such amino acids of a protein, polypeptide or peptide can often be substituted by one or more other such amino acids without eliminating a desired activity of that protein, polypeptide or peptide.

Thus the amino acids glycine, alanine, valine, leucine and isoleucine can often be substituted for one another (amino acids having aliphatic side chains). Of these possible substitutions it is preferred that glycine and alanine are used to substitute for one another (since they have relatively short side chains) and that valine, leucine and isoleucine are used to substitute for one another (since they have larger aliphatic side chains which are hydrophobic). Other amino acids which can often be substituted for one another include: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur containing side chains). It should be appreciated that amino acid substitutions within the scope of the present invention can be made using naturally occurring or non-naturally occurring amino acids. For example, it is contemplated herein that the methyl group on an alanine may be replaced with an ethyl group, and/or that minor changes may be made to the peptide backbone. Whether or not natural or synthetic amino acids are used, it is preferred that only L-amino acids are present.

Substitutions of this nature are often referred to as “conservative” or “semi-conservative” amino acid substitutions. The present invention therefore extends to use of a specific binding molecule comprising any of the amino acid sequence described above but with one or more conservative substitutions and or one or more tolerated substitutions in the sequence, such that the amino acid sequence of the variable domain has at least 90% identity, such as at least 90%, at least 91%, at least 92%, at least 93%, at least 94%, at least 95%, at least 96%, at least 97%, at least 98%, at least 99% or 100% identity, to the variable domain provided in SEQ ID NOS: 4, 5, or 6 and/or SEQ ID NOS: 7 or 8 or 9.

Identity” as known in the art is the relationship between two or more polypeptide sequences or two or more polynucleotide sequences, as determined by comparing the sequences. In the art, identity also means the degree of sequence relatedness between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic Acids Research, 12, 387 (1984), BLASTP, BLASTN, and FASTA (Atschul et al., J. Molec. Biol. 215, 403 (1990)).

One can use a program such as the CLUSTAL program to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of identity analysis are contemplated in the present invention.

The percent identity of two amino acid sequences or of two nucleic acid sequences is determined by aligning the sequences for optimal comparison purposes (e.g., gaps can be introduced in the first sequence for best alignment with the sequence) and comparing the amino acid residues or nucleotides at corresponding positions. The “best alignment” is an alignment of two sequences which results in the highest percent identity. The percent identity is determined by the number of identical amino acid residues or nucleotides in the sequences being compared (i.e., % identity=number of identical positions/total number of positions×100).

The determination of percent identity between two sequences can be accomplished using a mathematical algorithm known to those of skill in the art. An example of a mathematical algorithm for comparing two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. The BLASTn and BLASTp programs of Altschul, et al. (1990) J. Mol. Biol. 215:403-410 have incorporated such an algorithm. Determination of percent identity between two nucleotide sequences can be performed with the BLASTn program. Determination of percent identity between two protein sequences can be performed with the BLASTp program. To obtain gapped alignments for comparison purposes, Gapped BLAST can be utilised as described in Altschul et al. (1997) Nucleic Acids Res. 25:3389-3402. Alternatively, PSI-Blast can be used to perform an iterated search which detects distant relationships between molecules (Id.). When utilising BLAST, Gapped BLAST, and PSI-Blast programs, the default parameters of the respective programs (e.g., BLASTp and BLASTp) can be used. See http://www.ncbi.nlm.nih.gov. Default general parameters may include for example, Word Size=3, Expect Threshold=10. Parameters may be selected to automatically adjust for short input sequences. Another example of a mathematical algorithm utilised for the comparison of sequences is the algorithm of Myers and Miller, CABIOS (1989). The ALIGN program (version 2.0) which is part of the CGC sequence alignment software package has incorporated such an algorithm. Other algorithms for sequence analysis known in the art include ADVANCE and ADAM as described in Torellis and Robotti (1994) Comput. Appl. Biosci., 10 :3-5; and FASTA described in Pearson and Lipman (1988) Proc. Natl. Acad. Sci. 85:2444-8. Within FASTA, ktup is a control option that sets the sensitivity and speed of the search. For the purposes of evaluating percent identity in the present disclosure, BLASTp with the default parameters is used as the comparison methodology. In addition, when the recited percent identity provides a non-whole number value for amino acids (i.e., a sequence of 25 amino acids having 90% sequence identity provides a value of “22.5”, the obtained value is rounded down to the next whole number, thus “22”). Accordingly, in the example provided, a sequence having 22 matches out of 25 amino acids is within 90% sequence identity.

As will be obvious to those skilled in the art, it may be possible to truncate, or extend, the sequences provided at the C-terminus and/or N-terminus thereof, by 1, 2, 3, 4, 5 or more residues, without substantially affecting the functional characteristics of the specific binding molecule. All such variants are encompassed by the present invention.

Mutations, including conservative and tolerated substitutions, insertions and deletions, may be introduced into the sequences provided using any appropriate method including, but not limited to, those based on polymerase chain reaction (PCR), restriction enzyme-based cloning, or ligation independent cloning (LIC) procedures. These methods are detailed in many of the standard molecular biology texts. For further details regarding polymerase chain reaction (PCR) and restriction enzyme-based cloning, see Sambrook & Russell, (2001) Molecular Cloning—A Laboratory Manual (3^{rd} Ed.) CSHL Press. Further information on ligation independent cloning (LIC) procedures can be found in Rashtchian, (1995) Curr Opin Biotechnol 6(1): 30-6. The TCR sequences provided by the invention may be obtained from solid state synthesis, or any other appropriate method known in the art.

The specific binding molecules of the invention have the property of binding the KVLEYVIKV-HLA-A*02 complex. Specific binding molecules of the invention demonstrate a high degree of specificity for KVLEYVIKV-HLA-A*02 complex and are thus particularly suitable for therapeutic use. Specificity in the context of specific binding molecule of the invention relates to their ability to recognise HLA-A*02 target cells that are antigen positive, whilst having minimal ability to recognise HLA-A*02 target cells that are antigen negative.

As is known to those in the art the HLA-A*02 allele group includes a number of subtypes. All such subtypes are included within the scope of the invention. An example of a preferred subtype is HLA-A*0201.

Specificity can be measured in vitro, for example, in cellular assays such as those described in Examples 3 and 4. To test specificity, the binding molecules, such as TCRs, may be in soluble form and associated with an immune effector, and/or may be expressed on the surface of cells, such as T cells. Specificity may be determined by measuring the level of T cell activation in the presence of antigen positive and antigen negative target cells. Minimal recognition of antigen negative target cells is defined as a level of T cell activation of less than 20%, preferably less than 10%, preferably less than 5%, and more preferably less than 1%, of the level produced in the presence of antigen positive target cells, when measured under the same conditions and at a therapeutically relevant specific binding molecule concentration. For soluble TCRs associated with an immune effector, a therapeutically relevant concentration may be defined as a TCR concentration of 10⁻⁹ M or below, and/or a concentration of up to 100, preferably up to 1000, fold greater than the corresponding EC50 value. Preferably, for soluble TCRs associated with an immune effector there is at least a 100 fold difference in concentration required for T cell activation against antigen positive cells relative to antigen negative cells. Antigen positive cells may be obtained by peptide-pulsing using a suitable peptide concentration to obtain a level of antigen presentation comparable to cancer cells (for example, 10⁻⁹ M peptide, as described in Bossi et al., (2013) Oncoimmunol. 1; 2 (11):e26840) or, they may naturally present said peptide. Preferably, both antigen positive and antigen negative cells are human cells. Preferably antigen positive cells are human cancer cells. Antigen negative cells preferably include those derived from healthy human tissues.

Specificity may additionally, or alternatively, relate to the ability of a specific binding molecule to bind to KVLEYVIKV (SEQ ID NO: 1) HLA-A*02 complex and not to a panel of alternative peptide-HLA complexes. This may, for example, be determined by the Biacore method of Example 3. Said panel may contain at least 5, and preferably at least 10, alternative peptide-HLA -A*02 complexes. The alternative peptides may share a low level of sequence identity with SEQ ID NO: 1 and may be naturally presented. Alternative peptides are preferably derived from proteins expressed in healthy human tissues. Binding of the specific binding molecules to the KVLEYVIKV-HLA-A*02 complex may be at least 2 fold greater than to other naturally-presented peptide HLA complexes, more preferably at least 10 fold, or at least 50 fold or at least 100 fold greater, even more preferably at least 400 fold greater.

An alternative or additional approach to determine specific binding molecule specificity may be to identify the peptide recognition motif using sequential mutagenesis, e.g. alanine scanning. Residues that form part of the binding motif are those that are not permissible to substitution. Non-permissible substitutions may be defined as those peptide positions in which the binding affinity of the specific binding molecule is reduced by at least 50%, or preferably at least 80% relative to the binding affinity for the non-mutated peptide. Such an approach is further described in Cameron et al., (2013), Sci Transl Med. 2013 Aug. 7; 5 (197): 197ra103 and WO2014096803. Specificity in this case may be determined by identifying alternative motif containing peptides, particularly alternative motif containing peptides in the human proteome, and testing these peptides for binding to the specific binding molecule. Binding of the specific binding molecule to one or more alternative peptides may indicate a lack of specificity. In this case further testing of specificity via cellular assays may be required. A low tolerance for (alanine) substitutions in the central part of the peptide indicate that the specific binding molecule has a high specificity and therefore presents a low risk for cross-reactivity with alternative peptides.

Specific binding molecules of the invention may have an ideal safety profile for use as therapeutic reagents. In this case the specific binding molecules may be in soluble form and may preferably be fused to an immune effector. Suitable immune effectors include but are not limited to, cytokines, such as IL-2 and IFN-γ, superantigens and mutants thereof; chemokines such as IL-8, platelet factor 4, melanoma growth stimulatory protein; antibodies, including fragments, derivatives and variants thereof, that bind to antigens on immune cells such as T cells or NK cell (e.g. anti-CD3, anti-CD28 or anti-CD16); and complement activators. An ideal safety profile means that, in addition to demonstrating good specificity, the specific binding molecules of the invention may have passed further preclinical safety tests. Examples of such tests include whole blood assays to confirm minimal cytokine release in the presence of whole blood and thus low risk of causing a potential cytokine release syndrome in vivo, and alloreactivity tests to confirm low potential for recognition of alternative HLA types.

Specific binding molecules of the invention may be amenable to high yield purification. Yield may be determined based on the amount of material retained during the purification process (i.e. the amount of correctly folded material obtained at the end of the purification process relative to the amount of solubilised material obtained prior to refolding), and or yield may be based on the amount of correctly folded material obtained at the end of the purification process, relative to the original culture volume. High yield means greater than 1%, or more preferably greater than 5%, or higher yield. High yield means greater than 1 mg/ml, or more preferably greater than 3 mg/ml, or greater than 5 mg/ml, or higher yield.

Specific binding molecules of the invention preferably have a Ko for the KVLEYVIKV-HLA-A*02 complex of greater than (i.e. stronger than) the non-mutated, or scaffold TCR, for example in the range of 1 pM to 50 μM. In one aspect, specific binding molecules of the invention have a K_{D} for the complex of from about (i.e. +/−10%) 1 pM to about 400 nM, from about 1 pM to about 1000 pM, from about 100 pM to about 800 pM. Said specific binding molecules may additionally, or alternatively, have a binding half-life (T½) for the complex in the range of from about 1 min to about 60 h, from about 20 min to about 50 h, or from about 1 h to about 6 h. Preferably, specific binding molecules of the invention have a K_{D} for the KVLEYVIKV -HLA-A*02 complex of from about 200 pM to about 800 pM and/or a binding half-life from about 1 h to about 6 h. Such high-affinity is preferable for specific binding molecules associated with therapeutic agents and/or detectable labels.

In another aspect, specific binding molecules of the invention may have a Kp for the complex of from about 50 nM to about 200 μM, or from about 100 nM to about 1 μM and/or a binding half-life for the complex of from about 3 sec to about 12 min. Such specific binding molecules may be preferable for adoptive therapy applications.

Methods to determine binding affinity (inversely proportional to the equilibrium constant K_{D}) and binding half life (expressed as T½) are known to those skilled in the art. In a preferred embodiment, binding affinity and binding half-life are determined using Surface Plasmon Resonance (SPR) or Bio-Layer Interferometry (BLI), for example using a BIAcore instrument or Octet instrument, respectively. A preferred method is provided in Example 3. It will be appreciated that doubling the affinity of a specific binding molecules results in halving the K_{D}. T½ is calculated as In2 divided by the off-rate (k_{off}). Therefore, doubling of T½ results in a halving in k_{off}. K_{D} and k_{off} values for TCRs are usually measured for soluble forms of the TCR, i.e. those forms which are truncated to remove cytoplasmic and transmembrane domain residues. To account for variation between independent measurements, and particularly for interactions with dissociation times in excess of 20 hours, the binding affinity and or binding half-life of a given specific binding molecules may be measured several times, for example 3 or more times, using the same assay protocol, and an average of the results taken. To compare binding data between two samples (i.e. two different specific binding molecules and or two preparations of the same specific binding molecules) it is preferable that measurements are made using the same assay conditions (e.g. temperature), such as those described in Example 2.

Certain preferred specific binding molecules of the invention have a binding affinity for, and/or a binding half-life for, the KVLEYVIKV-HLA-A*02 complex that is substantially higher than that of the native TCR. Increasing the binding affinity of a native TCR often reduces the specificity of the TCR for its peptide-MHC ligand, and this is demonstrated in Zhao et al., (2007) J. Immunol, 179:9, 5845-5854. However, such specific binding molecules of the invention remain specific for the KVLEYVIKV-HLA-A*02 complex, despite having substantially higher binding affinity than the native TCR.

Certain preferred specific binding molecules are able to generate a highly potent T cell response in vitro against antigen positive cells, in particular those cells presenting low levels of antigen typical of cancer cells (i.e. in the order of 5-100, for example 50, antigens per cell (Bossi et al., (2013) Oncoimmunol. 1; 2 (11):e26840; Purbhoo et al., (2006). J Immunol 176(12): 7308-7316.)). Such specific binding molecules may be linked to an immune effector such as an anti-CD3 antibody. The T cell response that is measured may be the release of T cell activation markers such as Interferon γ or Granzyme B, or target cell killing, or other measure of T cell activation, such as T cell proliferation. Preferably a highly potent response is one with EC₅₀ value in the pM range, for example, 200 pM or lower.

Specific binding molecules of the invention may comprise TCR variable domains that can be αβ heterodimers. In certain cases, the specific binding molecules of the invention may comprise TCR variable domains that can be γδ heterodimers. In other cases, the specific binding molecules of the invention may comprise TCR variable domains that can be αα or ββ homodimers (or γγ or δδ homodimers). Alpha-beta heterodimeric specific binding molecules of the invention may comprise an alpha chain TRAC constant domain sequence and/or a beta chain TRBC1 or TRBC2 constant domain sequence. The constant domains may be full-length by which it is meant that extracellular, transmembrane and cytoplasmic domains are present, or they may be in soluble format (i.e. having no transmembrane or cytoplasmic domains). One or both of the constant domains may contain mutations, substitutions or deletions relative to the native TRAC and/or TRBC½ sequences. The term TRAC and TRBC½ also encompasses natural polymorphic variants, for example N to K at position 4 of TRAC (Bragado et al International immunology. 1994 February; 6(2):223-30). In addition, the N terminal amino acid of TRAC is most commonly N, but in some cases it may be D, or another amino acid. A preferred residue is D.

The alpha and beta chain constant domain sequences may be modified by truncation or substitution to delete the native disulphide bond between Cys4 of exon 2 of TRAC and Cys2 of exon 2 of TRBC1 or TRBC2. The alpha and/or beta chain constant domain sequence(s) may have an introduced disulphide bond between residues of the respective constant domains, as described, for example, in WO 03/020763. In a preferred embodiment the alpha and beta constant domains may be modified by substitution of cysteine residues at position Thr 48 of TRAC and position Ser 57 of TRBC1 or TRBC2, the said cysteines forming a disulphide bond between the alpha and beta constant domains of the TCR. TRBC1 or TRBC2 may additionally include a cysteine to alanine mutation at position 75 of the constant domain and an asparagine to aspartic acid mutation at position 89 of the constant domain. One or both of the extracellular constant domains present in an αβ heterodimer of the invention may be truncated at the C terminus or C termini, for example by up to 15, or up to 10, or up to 8 or fewer amino acids. One or both of the extracellular constant domains present in an αβ heterodimer of the invention may be truncated at the C terminus or C termini by, for example, up to 15, or up to 10 or up to 8 amino acids. The C terminus of the alpha chain extracellular constant domain may be truncated by 8 amino acids. Preferred examples of alpha and beta constant domain sequences are provided by SEQ ID NO: 10 and 11.

Alternatively, rather than full-length or truncated constant domains there may be no TCR constant domains. Accordingly, the specific binding molecule of the invention may be comprised of the variable domains of the TCR alpha and beta chains, optionally with additional domains as described herein. Additional domains include but are not limited to immune effector domains (such as antibody domains), Fc domains or albumin binding domains.

Specific binding molecules of the invention may be in single chain format. Single chain formats include, but are not limited to, αβ TCR polypeptides of the Vα-L-Vβ, Vβ-L-Vα, Vα-Cα-L-Vβ, Vα-L-Vβ-Cβ, or Vα-Cα-L-Vβ-Cβ types, wherein Vα and Vβ are TCR α and β variable regions respectively, Cα and Cβ are TCR α and β constant regions respectively, and L is a linker sequence (Weidanz et al., (1998) J Immunol Methods. December 1; 221(1-2):59-76; Epel et al., (2002), Cancer Immunol Immunother. November; 51(10):565-73; WO 2004/033685; WO9918129). Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length, The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used multi-domain binding molecules of the invention include, but are not limited to: GGGGS (SEQ ID No: 29), GGGSG (SEQ ID No: 30), GGSGG (SEQ ID No: 31), GSGGG (SEQ ID No: 32), GSGGGP (SEQ ID No: 33), GGEPS (SEQ ID No: 34), GGEGGGP (SEQ ID No: 35), and GGEGGGSEGGGS (SEQ ID No: 36) (as described in WO2010/133828) and GGGSGGGG (SEQ ID NO: 37). Additional linkers may include sequences having one or more of the following sequence motifs: GGGS (SEQ ID No: 38), GGGGS (SEQ ID No: 39), TVLRT (SEQ ID No: 40), TVSSAS (SEQ ID No: 41) and TVLSSAS (SEQ ID No: 42). Where present, one or both of the constant domains may be full length, or they may be truncated and/or contain mutations as described above. Preferably single chain TCRs are soluble. Certain single chain TCRs of the invention may have an introduced disulphide bond between residues of the respective constant domains, as described in WO 2004/033685. Single chain TCRs are further described in WO2004/033685; WO98/39482; WO01/62908; Weidanz et al. (1998) J Immunol Methods 221(1-2): 59-76; Hoo et al. (1992) Proc Natl Acad Sci USA 89(10): 4759-4763; Schodin (1996) Mol Immunol 33(9): 819-829). The TCR variable domains may be arranged in diabody format.

The invention also includes particles displaying specific binding molecules of the invention and the inclusion of said particles within a library of particles. Such particles include but are not limited to phage, yeast cells, ribosomes, or mammalian cells. Method of producing such particles and libraries are known in the art (for example see WO2004/044004; WO01/48145, Chervin et al. (2008) J. Immuno. Methods 339.2: 175-184).

Specific binding molecules of the invention are useful for delivering detectable labels or therapeutic agents to antigen presenting cells and tissues containing antigen presenting cells. They may therefore be associated (covalently or otherwise) with a detectable label (for diagnostic purposes wherein the specific binding molecule is used to detect the presence of cells presenting the cognate antigen); and/or a therapeutic agent; and/or a pharmacokinetic (PK) modifying moiety.

Examples of PK modifying moieties include, but are not limited to, PEG (Dozier et al., (2015) Int J Mol Sci. October 28; 16(10):25831-64 and Jevsevar et al., (2010) Biotechnol J.January; 5(1):113-28), PASylation (Schlapschy et al., (2013) Protein Eng Des Sel. August; 26(8):489-501), albumin, and albumin binding domains, (Dennis et al., (2002) J Biol Chem. September 20; 277(38):35035-43), and/or unstructured polypeptides (Schellenberger et al., (2009) Nat Biotechnol. December; 27(12):1186-90). Further PK modifying moieties include antibody Fc fragments. PK modifying moieties may serve to extend the in vivo half life.

Where an immunoglobulin Fc domain is used as a PK modifying moiety, it may be any antibody Fc region. The Fc region is the tail region of an antibody that interacts with cell surface Fc receptors and some proteins of the complement system. The Fc region typically comprises two polypeptide chains both having two or three heavy chain constant domains (termed CH2, CH3 and CH4), and a hinge region. The two chains being linked by disulphide bonds within the hinge region. Fc domains from immunoglobulin subclasses IgG1, IgG2 and IgG4 bind to and undergo FcRn mediated recycling, affording a long circulatory half-life (3-4 weeks). The interaction of IgG with FcRn has been localized in the Fc region covering parts of the CH2 and CH3 domain. Preferred immunoglobulin Fc for use in the present invention include, but are not limited to Fc domains from IgG1 or IgG4. Preferably the Fc domain is derived from human sequences. The Fc region may also preferably include KiH mutations which facilitate dimerization, as well as and mutations to prevent interaction with activating receptors i.e. functionally silent molecules. The immunoglobulin Fc domain may be fused to the C or N terminus of the other domains (i.e., the TCR variable domains or immune effector), in any suitable order or configuration. The immunoglobulin Fc may be fused to the other domains (i.e., the TCR variable domains or immune effector) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length, The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used multi-domain binding molecules of the invention include, but are not limited to: GGGGS (SEQ ID No: 29), GGGSG (SEQ ID No: 30), GGSGG (SEQ ID No: 31), GSGGG (SEQ ID No: 32), GSGGGP (SEQ ID No: 33), GGEPS (SEQ ID No: 34), GGEGGGP (SEQ ID No: 35), and GGEGGGSEGGGS (SEQ ID No: 36) (as described in WO2010/133828) and GGGSGGGG (SEQ ID NO: 37). Additional linkers may include sequences having one or more of the following sequence motifs: GGGS (SEQ ID No: 38), GGGGS (SEQ ID No: 39), TVLRT (SEQ ID No: 40), TVSSAS (SEQ ID No: 41) and TVLSSAS (SEQ ID No: 42). Where the immunoglobulin Fc is fused to the TCR, it may be fused to either the alpha or beta chains, with or without a linker. Furthermore, individual chains of the Fc may be fused to individual chains of the TCR.

Preferably the Fc region may be derived from the IgG1 or IgG4 subclass. The two chains may comprise CH2 and CH3 constant domains and all or part of a hinge region. The hinge region may correspond substantially or partially to a hinge region from IgG1, IgG2, IgG3 or IgG4. The hinge may comprise all or part of a core hinge domain and all or part of a lower hinge region. Preferably, the hinge region contains at least one disulphide bond linking the two chains.

The Fc region may comprise mutations relative to a WT sequence. Mutations include substitutions, insertions and deletions. Such mutations may be made for the purpose of introducing desirable therapeutic properties. For example, to facilitate heterodimersation, knobs into holes (KiH) mutations maybe engineered into the CH3 domain. In this case, one chain is engineered to contain a bulky protruding residue (i.e. the knob), such as Y, and the other is chain engineered to contain a complementary pocket (i.e. the hole). Suitable positions for KiH mutations are known in the art. Additionally or alternatively mutations may be introduced that abrogate or reduce binding to Fcy receptors and or increase binding to FcRn, and/or prevent Fab arm exchange, or remove protease sites.

The PK modifying moiety may also be an albumin-biding domain, which may also act to extend half-life. As is known in the art, albumin has a long circulatory half-life of 19 days, due in part to its size, being above the renal threshold, and by its specific interaction and recycling via FcRn. Attachment to albumin is a well-known strategy to improve the circulatory half-life of a therapeutic molecule in vivo. Albumin may be attached non-covalently, through the use of a specific albumin binding domain, or covalently, by conjugation or direct genetic fusion. Examples of therapeutic molecules that have exploited attachment to albumin for improved half-life are given in Sleep et al., Biochim Biophys Acta. 2013 December; 1830(12):5526-34.

The albumin-binding domain may be any moiety capable of binding to albumin, including any known albumin-binding moiety. Albumin binding domains may be selected from endogenous or exogenous ligands, small organic molecules, fatty acids, peptides and proteins that specifically bind albumin. Examples of preferred albumin binding domains include short peptides, such as described in Dennis et al., J Biol Chem. 2002 Sept. 20; 277(38):35035-43 (for example the peptide QRLMEDICLPRWGCLWEDDF); proteins engineered to bind albumin such as antibodies, antibody fragments and antibody like scaffolds, for example Albudab® (O'Connor-Semmes et al., Clin Pharmacol Ther. 2014 December; 96(6):704-12), commercially provided by GSK and Nanobody® (Van Roy et al., Arthritis Res Ther. 2015 May 20; 17:135), commercially provided by Ablynx; and proteins based on albumin binding domains found in nature such as Streptococcal protein G Protein (Stork et al., Eng Des Sel. 2007 November; 20(11):569-76), for example Albumod® commercially provided by Affibody Preferably, albumin is human serum albumin (HSA). The affinity of the albumin binding domain for human albumin may be in the range of picomolar to micromolar. Given the extremely high concentration of albumin in human serum (35-50 mg/ml, approximately 0.6 mM), it is calculated that substantially all of the albumin binding domains will be bound to albumin in vivo.

The albumin-binding moiety may be linked to the C or N terminus of the other domains (i.e., the TCR variable domains or immune effector) in any suitable order or configuration. The albumin-binding moiety may be linked to the other domains (i.e., the TCR variable domains or immune effector) via a linker. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. The linker may be 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids in length. Examples of suitable linkers that may be used in multi-domain binding molecules of the invention include, but are not limited to: GGGGS (SEQ ID No: 29), GGGSG (SEQ ID No: 30), GGSGG (SEQ ID No: 31), GSGGG (SEQ ID No: 32), GSGGGP (SEQ ID No: 33), GGEPS (SEQ ID No: 34), GGEGGGP (SEQ ID No: 35), and GGEGGGSEGGGS (SEQ ID No: 36) (as described in WO2010/133828) and GGGSGGGG (SEQ ID NO: 37). Additional linkers may include sequences having one or more of the following sequence motifs: GGGS (SEQ ID No: 38), GGGGS (SEQ ID No: 39), TVLRT (SEQ ID No: 40), TVSSAS (SEQ ID No: 41) and TVLSSAS (SEQ ID No: 42). Where the albumin-binding moiety is linked to the TCR, it may be linked to either the alpha or beta chains, with or without a linker.

Detectable labels for diagnostic purposes include for instance, fluorescent labels, radiolabels, enzymes, nucleic acid probes and contrast reagents.

For some purposes, the specific binding molecules of the invention may be aggregated into a complex comprising several specific binding molecules to form a multivalent complex. There are a number of human proteins that contain a multimerisation domain that may be used in the production of multivalent complexes. For example the tetramerisation domain of p53 which has been utilised to produce tetramers of scFv antibody fragments which exhibited increased serum persistence and significantly reduced off-rate compared to the monomeric scFv fragment (Willuda et al. (2001) J. Biol. Chem. 276 (17) 14385-14392). Haemoglobin also has a tetramerisation domain that could be used for this kind of application. A multivalent complex of the invention may have enhanced binding capability for the complex compared to a non-multimeric complex. Thus, multivalent complexes of are also included within the invention. Such multivalent complexes are particularly useful for tracking or targeting cells presenting particular antigens in vitro or in vivo.

Therapeutic agents which may be associated with the specific binding molecules of the invention include immune-modulators and effectors, radioactive compounds, enzymes (perforin for example) or chemotherapeutic agents (cis-platin for example). To ensure that toxic effects are exercised in the desired location the toxin could be inside a liposome linked to the specific binding molecules so that the compound is released slowly. This will prevent damaging effects during the transport in the body and ensure that the toxin has maximum effect after binding of the specific binding molecule to the relevant antigen presenting cells.

Examples of suitable therapeutic agents include, but are not limited to:
small molecule cytotoxic agents, i.e. compounds with the ability to kill mammalian cells having a molecular weight of less than 700 Daltons. Such compounds could also contain toxic metals capable of having a cytotoxic effect. Furthermore, it is to be understood that these small molecule cytotoxic agents also include pro-drugs, i.e. compounds that decay or are converted under physiological conditions to release cytotoxic agents. Examples of such agents include cis-platin, maytansine derivatives, rachelmycin, calicheamicin, docetaxel, etoposide, gemcitabine, ifosfamide, irinotecan, melphalan, mitoxantrone, sorfimer sodiumphotofrin II, temozolomide, topotecan, trimetreate 21arbour21ate, auristatin E vincristine and doxorubicin;
peptide cytotoxins, i.e. proteins or fragments thereof with the ability to kill mammalian cells. For example, ricin, diphtheria toxin, pseudomonas bacterial exotoxin A, Dnase and Rnase;
radio-nuclides, i.e. unstable isotopes of elements which decay with the concurrent emission of one or more of α or β particles, or γ rays. For example, iodine 131, rhenium 186, indium 111, yttrium 90, bismuth 210 and 213, actinium 225 and astatine 213; chelating agents may be used to facilitate the association of these radio-nuclides to the high affinity TCRs, or multimers thereof;
Immuno-stimulants, i.e. immune effector molecules which stimulate immune response. For example, cytokines such as IL-2 and IFN-γ,
Superantigens and mutants thereof;
TCR-HLA fusions, e.g. fusion to a peptide-HLA complex, wherein said peptide is derived from a common human pathogen, such as Epstein Barr Virus (EBV);
chemokines such as IL-8, platelet factor 4, melanoma growth stimulatory protein, etc;
antibodies or fragments thereof, including anti-T cell or NK cell determinant antibodies (e.g. anti-CD3, anti-CD28 or anti-CD16);
alternative protein scaffolds with antibody like binding characteristics
complement activators;
xenogeneic protein domains, allogeneic protein domains, viral/bacterial protein domains, viral/bacterial peptides.

In a preferred aspect, the specific binding molecules comprise an immune effector domain, usually by fusion to said immune effector to the N-or C-terminus of the alpha or beta chain, or both, in any suitable configuration, of the specific binding molecule. A particularly preferred immune effector is an anti-CD3 antibody, or a functional fragment or variant of said anti-CD3 antibody. As used herein, the term “antibody” encompasses such fragments and variants. Examples of anti-CD3 antibodies include but are not limited to OKT3, UCHT-1, BMA-031 and 12F6. Antibody fragments and variants/analogues which are suitable for use in the compositions and methods described herein include minibodies, Fab fragments, F(ab′)₂ fragments, dsFv and scFv fragments, Nanobodies™ (these constructs, marketed by Ablynx (Belgium), comprise synthetic single immunoglobulin variable heavy domain derived from a camelid (e.g. camel or llama) antibody) and Domain Antibodies (Domantis (Belgium), comprising an affinity matured single immunoglobulin variable heavy domain or immunoglobulin variable light domain) or alternative protein scaffolds that exhibit antibody like binding characteristics such as Affibodies (Affibody (Sweden), comprising engineered protein A scaffold) or Anticalins (Pieris (Germany)), comprising engineered anticalins) to name but a few. In a preferred embodiment the anti-CD3 is an scFv fragment corresponding to SEQ ID NO: 12-14.

In another preferred format the variable domains and immune effector domains of the specific binding molecule may be alternated on separate polypeptide chains, leading to dimerization. Such formats are described for example in WO2019012138. In brief, the first polypeptide chain could include (from N to C terminus) a first antibody variable domain followed by a TCR variable domain, optionally followed by a Fc domain. The second chain could include (from N to C terminus) a TCR variable domain followed by a second antibody variable domain, optionally followed by a Fc domain. Given linkers of an appropriate length, the chains would dimerise into a multi-specific molecule, optionally including a Fc domain. Molecules in which domains are located on different chains in this way may also be referred to as diabodies, which are also contemplated herein. Additional chains and domains may be added to form, for example, triabodies.

Accordingly, there is also provided herein a specific binding molecule selected from the group of molecules comprising a first polypeptide chain and a second polypeptide chain, wherein: the first polypeptide chain comprises a first binding region of a variable domain (VD1) of an antibody specifically binding to a cell surface antigen of a human immune effector cell, and
a first binding region of a variable domain (VR1) of a TCR specifically binding to an MHC-associated peptide epitope, and
a first linker (LINK1) connecting said domains;
the second polypeptide chain comprises a second binding region of a variable domain (VR2) of a TCR specifically binding to an MHC-associated peptide epitope, and
a second binding region of a variable domain (VD2) of an antibody specifically binding to a cell surface antigen of a human immune effector cell, and
a second linker (LINK2) connecting said domains;
wherein said first binding region (VD1) and said second binding region (VD2) associate to form a first binding site (VD1) (VD2) that binds a cell surface antigen of a human immune effector cell;
said first binding region (VR1) and said second binding region (VR2) associate to form a second binding site (VR1) (VR2) that binds said MHC-associated peptide epitope;
wherein said two polypeptide chains are fused to human lgG hinge domains and/or human IgG Fc domains or dimerizing portions thereof; and
wherein the said two polypeptide chains are connected by covalent and/or non-covalent bonds between said hinge domains and/or Fc-domains; and
wherein said dual specificity polypeptide molecule is capable of simultaneously binding the cell surface molecule and the MHC-associated peptide epitope, and dual specificity polypeptide molecules, wherein the order of the binding regions in the two polypeptide chains is selected from VD1-VR1 and VR2-VD2 or VD1-VR2 and VR1-VD2, or VD2-VR1 and VR2-VD1 or VD2-VR2 and VR1-VD1 and wherein the domains are either connected by LINK1 or LINK2, wherein the MHC-associated peptide epitope is KVLEYVIKV and the MHC is HLA-A*02.

Linkage of the TCR and the anti-CD3 antibody may be via covalent or non-covalent attachment. Covalent attachment may be direct, or indirect via a linker sequence. Linker sequences are usually flexible, in that they are made up primarily of amino acids such as glycine, alanine and serine, which do not have bulky side chains likely to restrict flexibility. Alternatively, linkers with greater rigidity may be desirable. Usable or optimum lengths of linker sequences may be easily determined. Often the linker sequence will be less than about 12, such as less than 10, or from 2-10 amino acids in length. Examples of suitable linkers that may be used in TCRs of the invention include, but are not limited to: GGGGS (SEQ ID No: 29), GGGSG (SEQ ID No: 30), GGSGG (SEQ ID No: 31), GSGGG (SEQ ID No: 32), GSGGGP (SEQ ID No: 33), GGEPS (SEQ ID No: 34), GGEGGGP (SEQ ID No: 35), and GGEGGGSEGGGS (SEQ ID No: 36) (as described in WO2010/133828) and GGGSGGGG (SEQ ID NO: 37

Preferred specific binding molecule-anti-CD3 fusion constructs of the invention include those in which the alpha chain variable domain comprises the amino acid sequence of any one or SEQ ID NOs: 4-6 or a sequence having at least 90% identity thereto and/or the beta chain variable domain comprises the amino acid sequence of any one of SEQ ID NOs: 7-9 or a sequence having at least 90% identity thereto. Said alpha and beta chains may further comprise an alpha and beta extracellular constant region comprising a non-native disulphide bond. The constant domain of the alpha chain may be truncated by eight amino acids. The alpha and beta extracellular constant regions may be provided by SEQ ID NOS 10 and 11 respectively. An anti-CD3 scFv antibody fragment selected from SEQ ID NOS 12-14 may be fused to the N terminus of the beta chain via a linker, which may be selected from SEQ ID NOs: 29-32.

Particularly preferred sequences of anti-CD3-TCR fusion constructs of the invention are provided by:
Alpha variable domain SEQ ID NO 4 and constant domain SEQ ID No 10,
Beta variable domain SEQ ID NO 8 and constant domain SEQ ID No 11,
The anti-CD3 scFv antibody fragment of SEQ ID NO 12 fused to the N terminus of the beta chain via the linker of SEQ ID NO: 29.
   or
Alpha variable domain SEQ ID NO 4 and constant domain SEQ ID No 10,
Beta variable domain SEQ ID NO 8 and constant domain SEQ ID No 11,
The anti-CD3 scFv antibody fragment of SEQ ID NO 14 fused to the N terminus of the beta chain via the linker of SEQ ID NOs: 29

Also included within the scope of the invention are functional variants of said anti-CD3-TCR fusion constructs. Said functional variants preferably have at least 90% identity, such as at least 90%, at least 91%, at least 92%, at least 93%, at least 94%, at least 95%, at least 96%, at least 97%, at least 98%, at least 99% or 100% identity to the above sequences, but are nonetheless functionally equivalent.

In a further aspect, the present invention provides nucleic acid encoding a alpha chain and/or a beta chain of a specific binding molecule, or specific binding molecule anti-CD3 fusion of the invention. In some embodiments, the nucleic acid is cDNA. In some embodiments the nucleic acid may be mRNA. In some embodiments, the invention provides nucleic acid comprising a sequence encoding an a chain variable domain of a specific binding molecule of the invention. In some embodiments, the invention provides nucleic acid comprising a sequence encoding a β chain variable domain of a specific binding molecule of the invention. The nucleic acid may be non-naturally occurring and/or purified and/or engineered. The nucleic acid sequence may be codon optimised, in accordance with expression system utilised. As is known to those skilled in the art, expression systems may include bacterial cells such as E. coli, or yeast cells, or mammalian cells, or insect cells, or they may be cell free expression systems.

In another aspect, the invention provides a vector which comprises nucleic acid of the invention. Preferably the vector is a TCR expression vector. Suitable TCR expression vectors include, for example, gamma-retroviral vectors or, more preferably, lentiviral vectors. Further details can be found in Zhang 2012 and references therein (Zhang et al,. Adv Drug Deliv Rev. 2012 Jun. 1; 64(8): 756-762).

The invention also provides a cell harbouring a vector of the invention, preferably a TCR expression vector. Suitable cells include, mammalian cells, preferably immune cells, even more preferably T cells. The vector may comprise nucleic acid of the invention encoding in a single open reading frame, or two distinct open reading frames, encoding the alpha chain and the beta chain respectively. Another aspect provides a cell harbouring a first expression vector which comprises nucleic acid encoding the alpha chain of a specific binding molecule of the invention, and a second expression vector which comprises nucleic acid encoding the beta chain of a specific binding molecule of the invention. Such cells are particularly useful in adoptive therapy. The cells of the invention may be isolated and/or recombinant and/or non-naturally occurring and/or engineered.

Since the specific binding molecules of the invention have utility in adoptive therapy, the invention includes a non-naturally occurring and/or purified and/or or engineered cell, especially a T-cell, presenting a specific binding molecule of the invention. The invention also provides an expanded population of T cells presenting a specific binding molecule, such as a TCR, of the invention. There are a number of methods suitable for the transfection of T cells with nucleic acid (such as DNA, cDNA or RNA) encoding the specific binding molecules of the invention (see for example Robbins et al., (2008) J Immunol. 180: 6116-6131). T cells expressing the specific binding molecules of the invention will be suitable for use in adoptive therapy-based treatment of cancer. As will be known to those skilled in the art, there are a number of suitable methods by which adoptive therapy can be carried out (see for example Rosenberg et al., (2008) Nat Rev Cancer 8(4): 299-308).

As is well-known in the art, TCRs may be subject to post translational modifications. Glycosylation is one such modification, which comprises the covalent attachment of oligosaccharide moieties to defined amino acids in the TCR chain. For example, asparagine residues, or serine/threonine residues are well-known locations for oligosaccharide attachment. The glycosylation status of a particular protein depends on a number of factors, including protein sequence, protein conformation and the availability of certain enzymes. Furthermore, glycosylation status (i.e. oligosaccharide type, covalent linkage and total number of attachments) can influence protein function. Therefore, when producing recombinant proteins, controlling glycosylation is often desirable. Controlled glycosylation has been used to improve antibody based therapeutics. (Jefferis et al., (2009) Nat Rev Drug Discov March; 8(3):226-34.). For soluble specific binding molecules of the invention glycosylation may be controlled, by using particular cell lines for example (including but not limited to mammalian cell lines such as Chinese hamster ovary (CHO) cells or human embryonic kidney (HEK) cells), or by chemical modification. Such modifications may be desirable, since glycosylation can improve pharmacokinetics, reduce immunogenicity and more closely mimic a native human protein (Sinclair and Elliott, (2005) Pharm Sci. August; 94(8):1626-35).

For administration to patients, the specific binding molecules of the invention (preferably associated with a detectable label or therapeutic agent or expressed on a transfected T cell), specific binding molecule-anti CD3 fusion molecules, nucleic acids, expression vectors or cells of the invention may be provided as part of a sterile pharmaceutical composition together with one or more pharmaceutically acceptable carriers or excipients. This pharmaceutical composition may be in any suitable form, (depending upon the desired method of administering it to a patient). It may be provided in unit dosage form, will generally be provided in a sealed container and may be provided as part of a kit. Such a kit would normally (although not necessarily) include instructions for use. It may include a plurality of said unit dosage forms.

The pharmaceutical composition may be adapted for administration by any appropriate route, such as parenteral (including subcutaneous, intramuscular, intrathecal or intravenous), enteral (including oral or rectal), inhalation or intranasal routes. Such compositions may be prepared by any method known in the art of pharmacy, for example by mixing the active ingredient with the carrier(s) or excipient(s) under sterile conditions.

Dosages of the substances of the present invention can vary between wide limits, depending upon the disease or disorder to be treated, the age and condition of the individual to be treated, etc. a suitable dose range for a TCR-anti-CD3 fusion molecules may be in the range of 25 ng/kg to 50 μg/kg or 1 μg to 1 g. A physician will ultimately determine appropriate dosages to be used.

Specific binding molecules, specific binding molecule-anti-CD3 fusion molecules, pharmaceutical compositions, vectors, nucleic acids and cells of the invention may be provided in substantially pure form, for example, at least 80%, at least 85%, at least 90%, at least 91%, at least 92%, at least 93%, at least 94%, at least 95%, at least 96%, at least 97%, at least 98%, at least 99% or 100% pure.

Also provided by the invention are:
A specific binding molecule, specific binding molecule-anti-CD3 fusion molecule, nucleic acid, pharmaceutical composition or cell of the invention for use in medicine, preferably for use in a method of treating cancer or a tumour;
the use of a specific binding molecule, specific binding molecule-anti-CD3 fusion molecule, nucleic acid, pharmaceutical composition or cell of the invention in the manufacture of a medicament for treating cancer or a tumour;
a method of treating cancer or a tumour in a patient, comprising administering to the patient a specific binding molecule, specific binding molecule-anti-CD3 fusion molecule, nucleic acid, pharmaceutical composition or cell of the invention;
an injectable formulation for administering to a human subject comprising a specific binding molecule, specific binding molecule-anti-CD3 fusion molecule, nucleic acid, pharmaceutical composition or cell of the invention.

The cancer may be a solid or liquid tumour. Preferably the tumour expresses MAGEA1. Examples of MAGEA1 expressing tumours include but are not limited to liver (HCC); lung (NSCLC and SCLC); bladder, head and neck, gastric, esophageal, breast, cutaneous melanoma, ovarian, cervical, endometrial and multiple myeloma. The specific binding molecule, specific binding molecule-anti-CD3 fusion molecule, nucleic acid, pharmaceutical composition or cell of the invention may be administered by injection, such as intravenous or subcutaneous or direct intratumoral injection. The human subject may be of the HLA-A*02 subtype.

The method of treatment may further include administering separately, in combination, or sequentially, an additional anti-neoplastic agent. Example of such agents are known in the art and may include immune activating agents and/or T cell modulating agents.

The invention also provides a method of producing a specific binding molecule of the invention or a specific binding molecule-anti-CD3 fusion molecule of the invention, comprising a) maintaining a cell according to the invention under optimal conditions for expression of the specific binding molecule chains and b) isolating the specific binding molecule chains.

Preferred features of each aspect of the invention are as for each of the other aspects mutatis mutandis. The prior art documents mentioned herein are incorporated by reference to the fullest extent permitted by law.

### DESCRIPTION OF THE DRAWINGS

FIG. 1—provides amino acid sequences of alpha and beta chain variable regions. CDRs are underlined

FIG. 2—provides amino acid sequences of mutated TCR alpha and beta chain variable regions. CDRs are underlined and mutations relative to WT are shown in bold.

FIG. 3—provides the amino acid sequences of alpha and beta chain extracellular constant regions

FIG. 4—provides the amino acid sequences of anti-CD3 scFv variants

FIG. 5—demonstrates potent T cell activation mediated by TCR-anti-CD3 fusions proteins against antigen positive target cells as described in Example 3

FIG. 6—demonstrates minimal recognition of cells derived from normal tissues by TCR-anti-CD3 fusions proteins as described in Example 3

FIG. 7—demonstrates killing of tumour cells by TCR-anti-CD3 fusions proteins as described in Example 4

The invention is further described in the following non-limiting examples.

### EXAMPLES

### Example 1—Expression, Refolding and Purification of Soluble TCRs and TCR-anti-CD3 Fusions Proteins

### Method

DNA sequences encoding the alpha and beta extracellular regions of a soluble TCR or TCR-antiCD3 fusion proteins were cloned separately into pGMT7-based expression plasmids using standard methods (as described in Sambrook, et al. Molecular cloning. Vol. 2. (1989) New York: Cold spring harbour laboratory press). The expression plasmids were transformed separately into E. coli strain Rosetta (BL21pLysS). For expression, cells were grown in auto-induction media supplemented with 1% glycerol (+ampicillin 100 μg/ml and 34 μg/ml chloramphenicol) at 230 rpm at 37C for 2 hours before reducing the temperature to 30° C. overnight. Cells were subsequently harvested by centrifugation. Cell pellets were lysed with BugBuster protein extraction reagent (Merck Millipore) according to the manufacturer's instructions. Inclusion body pellets were recovered by centrifugation. Pellets were washed twice in Triton buffer (50 mM Tris-HCl pH 8.1, 0.5% Triton-X100, 100 mM NaCl, 10 mM NaEDTA) and finally resuspended in detergent free buffer (50 mM Tris-HCl PH 8.1, 100 mM NaCl, 10 mM NaEDTA). Inclusion body protein yield was quantified by solubilising with 6 M guanidine-HCl and measuring OD₂₈₀. Protein concentration was then calculated using the extinction coefficient. Inclusion body purity was measured by solubilising with 8 M Urea and loading ˜2 μg onto 4-20% SDS-PAGE under reducing conditions. Purity was then estimated or calculated using densitometry software (Chemidoc, Biorad). Inclusion bodies were stored at +4° C. for short term storage and at −20° C. or −70° C. for longer term storage.

For soluble TCR refolding, α and β chain-containing inclusion bodies were first mixed and diluted into solubilisation/denaturation buffer (6 M Guanidine-hydrochloride, 50 mM Tris HCl PH 8.1, 100 mM NaCl, 10 mM EDTA, 20 mM DTT) followed by incubation for 30 min at 37° C. Refolding was then initiated by further dilution into refold buffer (100 mM Tris pH 8.1, 800 or 400 mM L-Arginine HCL, 2 mM EDTA, 4 M Urea, 6.5 mM cysteamine hydrochloride and 1.9 mM cystamine dihydrochloride) and the solution mixed well. The refolded mixture was dialysed against 10 L H₂O per L of refold for 18-20 hours at 5° C.+3° C. After this time, the dialysis buffer was twice replaced with10 mM Tris pH 8.1 (10 L) and dialysis continued for another 15 hours. The refold mixture was then filtered through 0.45 μm cellulose filters. Preparation of TCR-antiCD3 fusion molecules was carried out as described except that concentration of the redox reagents in the refolding step were 1 mM cystamine dihydrochloride, 10 mM cysteamine hydrochloride).

Purification of soluble TCRs was initiated by applying the dialysed refold onto a POROS® 50HQ anion exchange column and eluting bound protein with a gradient of 0-500 mM NaCl in 20 mM Tris pH 8.1 over 6 column volumes using an Akta® Pure (GE Healthcare). Peak TCR fractions were identified by SDS PAGE before being pooled and concentrated. The concentrated sample was then applied to a Superdex® 200 Increase 10/300 GL gel filtration column (GE Healthcare) pre-equilibrated in Dulbecco's PBS buffer. The peak TCR fractions were pooled and concentrated and the final yield of purified material calculated. For TCR-antiCD3 fusion molecules a cation exchange step was added following the anion exchange step. In this case, the peak fractions from anion exchange were diluted 20-fold in 40 mM MES, and applied to a POROS® 50HS cation exchange column. Bound protein was eluted with a gradient of 0-500 mM NaCl in 40 mM MES. Peak fractions were pooled and adjusted to 200 mM Tris pH 8.1, before being concentrated and applied directly to the gel filtration matrix as described.

### Example 2—Binding Characterisation

Binding analysis of purified soluble TCRs and TCR-antiCD3 fusion molecules to peptide-HLA complex was carried out by surface plasmon resonance, using either a BIAcore 8K, BIAcore 3000 or BIAcore T200 instrument. Biotinylated class I HLA-A*02 molecules were refolded with the peptide of interest and purified using methods known to those in the art (O'Callaghan et al. (1999). Anal Biochem 266(1): 9-15; Garboczi, et al. (1992). Proc Natl Acad Sci USA 89(8): 3429-3433). All measurements were performed at 25° C. in Dulbecco's PBS buffer, supplemented with 0.005% P20.

### BIAcore Method

Biotinylated peptide-HLA monomers were immobilized on to streptavidin-coupled CM-5 of Biotin CAPture sensor chips. Equilibrium binding constants were determined using serial dilutions of soluble TCR or fusion molecules injected at a constant flow rate of 10-30 μl min⁻¹ over a flow cell coated with ˜500 response units (RU) of peptide-HLA-A*02 complex. Equilibrium responses were normalised for each TCR concentration by subtracting the bulk buffer response on a control flow cell containing no peptide-HLA. The Ko value was obtained by non-linear curve fitting using Prism software and the Langmuir binding isotherm, bound=C*Max/(C+KD), where “bound” is the equilibrium binding in RU at injected TCR concentration C and Max is the maximum binding.

For high affinity interactions, binding parameters were determined by single cycle kinetics analysis. Five different concentrations of soluble TCR or fusion protein were injected over a flow cell coated with ˜50-200 RU of peptide-HLA complex using a flow rate of 50-60 μl min⁻¹. Typically, 60-200 μl of soluble TCR or fusion molecule was injected at a top concentration of between 2-100 nM , with successive 2 fold dilutions used for the other four injections. The lowest concentration was injected first. To measure the dissociation phase, buffer was injected until ≥ 10% dissociation occurred, typically after 1-3 hours. Kinetic parameters were calculated using the manufacturer's software. The dissociation phase was fitted to a single exponential decay equation enabling calculation of half-life. The equilibrium constant K_{D} was calculated from k_{off}/kₒₙ.

### Results

### Binding Characterisation of Soluble WT TCR

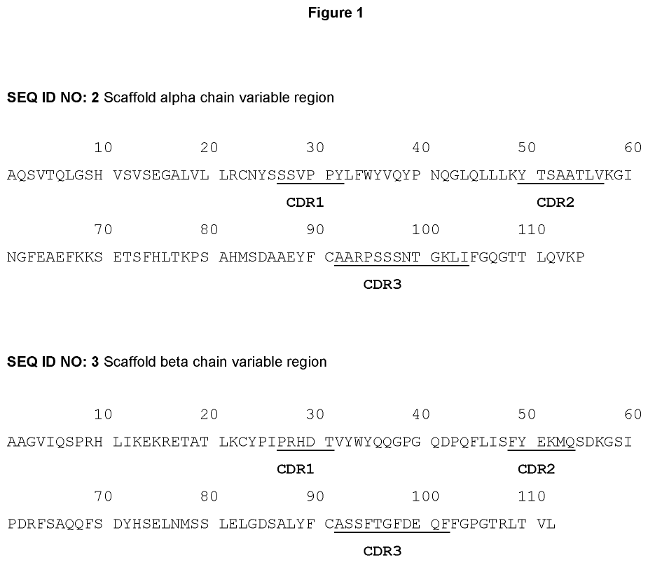

Soluble WT TCR comprising the alpha and beta variable domains provided in FIG. 1 and the alpha and beta extracellular constant domains provided in FIG. 3 was shown to bind KVLEYVIKV-HLA-A*02 complex with a K_{D} of 21.6 μM +/−3 μM (n=2).

The same soluble TCR was assessed for binding against a panel of 24 irrelevant peptide HLA-A*02 complexes. The irrelevant pHLAs were divided into three groups and loaded onto one of three flow cells. Soluble wild type TCR was injected at concentrations of 117 and 21.6 μM over all flow cells. No significant binding was detected at either concentration indicting that the soluble WT TCR is specific for the-HLA-A*02 complex.

Additional specificity assessment was carried out using a panel of peptides in which each residue of the KVLEYVIKV peptide was sequentially replaced with alanine. Relative binding to each of the alanine substituted peptides was determined. It was shown that alanine substitutions in the central part of the peptide result in complete loss of TCR binding (positions E4, Y5 and V6). These data indicate that the TCR it is particularly suitable for development of therapeutic reagents.

### Binding Characterisation of TCR-AntiCD3 Fusion Proteins

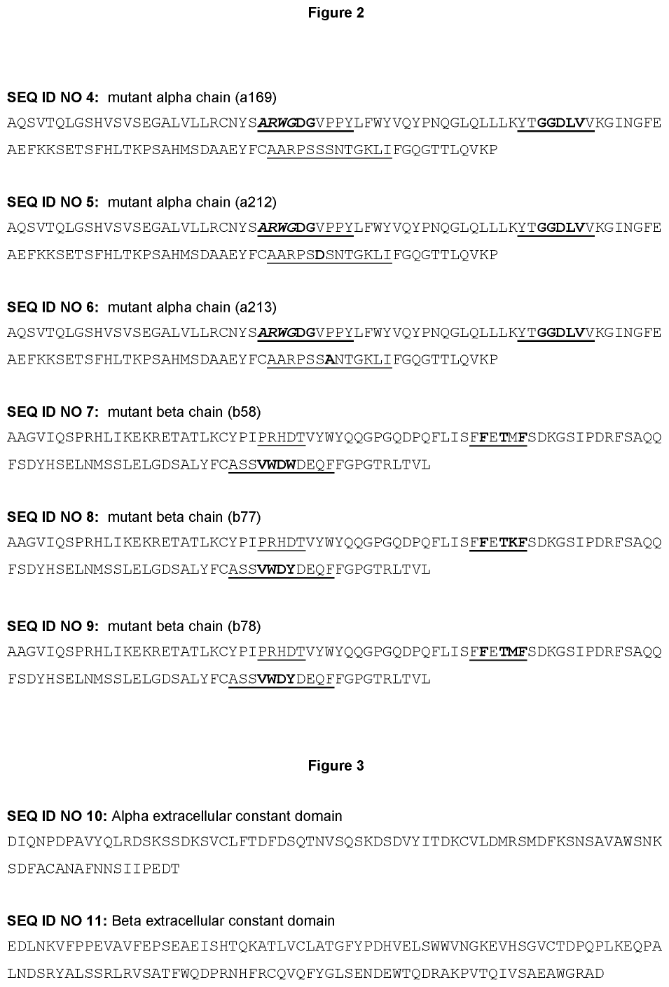

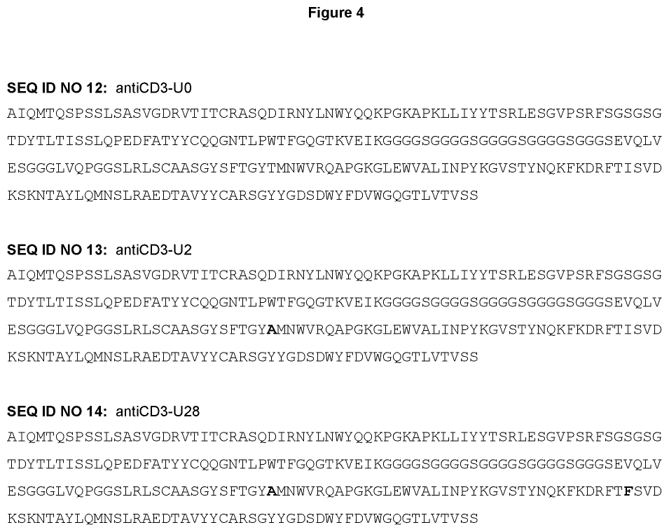

TCR-antiCD3 fusion proteins were prepared comprising the mutated variable domains provided in FIG. 2, the extracellular constant domains provided in FIG. 3 and the antiCD3 scFv sequences provided in FIG. 4. In this example, the anti-CD3 was fused to the N terminus of the beta variable domain in each case. Binding parameters of various fusions to KVLEYVIKV-HLA-A*02 complex are shown in the table below.

| Alpha chain | Beta chain | K_{D}(nM) | T_{1/2} (h) |
|---|---|---|---|
| a169 | b58 | 0.628 | 1 |
| a169 | b77 | 0.645 | 2.1 |
| a212 | b58 | 0.460 | 1.8 |
| a212 | b78 | 0.409 | 1.1 |
| a213 | b78 | 0.115 | 2.9 |

### Example 3—Potent and Specific T Cell Activation Against Antigen Positive Target Cells

### T Cell Activation

TCR-antiCD3 fusion proteins were assessed for their ability to mediate potent and specific activation of CD3+ T cells against cells presenting the KVLEYVIKV-HLA-A*02 complex. Interferon-γ (IFN-γ) release was used as a read out for T cell activation.

### Method

Assays were performed using a human IFN-γ ELISPOT kit (BD Biosciences) according to the manufacturer's instructions. Briefly, ELISpot plates were coated with IFNy antibody 1-7 days before. On the day of the assay, ELISPOT plates were blocked with 100 μl assay medium (R10). After removal of block, target cells were plated at 50,000/well in 50 μl. Fusion protein were titrated to give final concentrations of 5, 1, 0.5, 0.3, 0.2, 0.1, 0.05, 0.03, 0.02 and 0.01 nM (spanning the anticipated clinically relevant range), and added to the well in a volume of 50 μl. Effectors (PBMCs) were thawed from liquid nitrogen counted and plated at 20-80,000 cells/well in 50 μl (the exact number of cells used for each experiment is donor dependent and may be adjusted to produce a response within a suitable range for the assay). The final volume of each well was made up to 200 ul with R10. The plates/cells were cultured overnight and the next day the plates were developed and allowed to dry at room temperature for at least 2 hours prior to counting the spots using a CTL analyser with Immunospot software (Cellular Technology Limited). Response curves were plotted using PRISM and Ec50 values were calculated.

In this example, the following human cancer cells lines were used as target cells:
NCI-H1703 (antigen positive) lung
NCI-H2023 (antigen positive) lung
U-2-OS (antigen positive) bone
MeI624 (antigen negative) melanoma

### Results

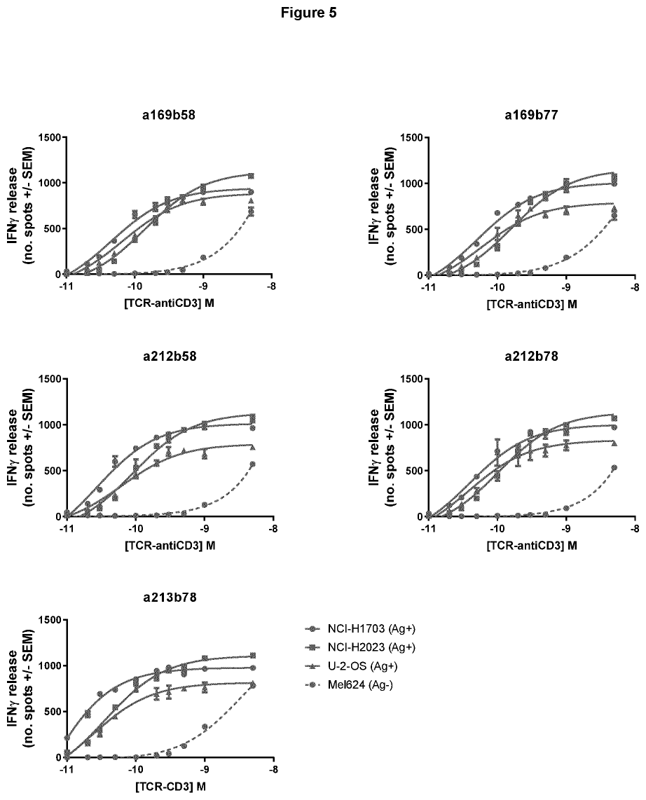

FIG. 5 shows response curves generated using TCR-antiCD3 fusion proteins of the invention. In each case Ec50 values were in the low pM range (<200 pM) against antigen positive cells. Only minimal activation was observed against antigen negative cells within a therapeutically relevant range (≤1 nM), resulting in a wide safety window between on-target and off-target stimulation.

### Minimal Recognition of Normal Tissue

To further demonstrate the specificity of TCR-antiCD3 fusion proteins, further testing was carried out using the same ELISPOT methodology as described above, using a panel of normal cells derived from healthy human tissues as targets. Cancer cell lines NCI-H1703 and NCI-H2087 were used as antigen positive and antigen negative controls respectively. Fusion protein were titrated to give final concentrations of 2, 1, 0.5, and 0.2 nM

### Results

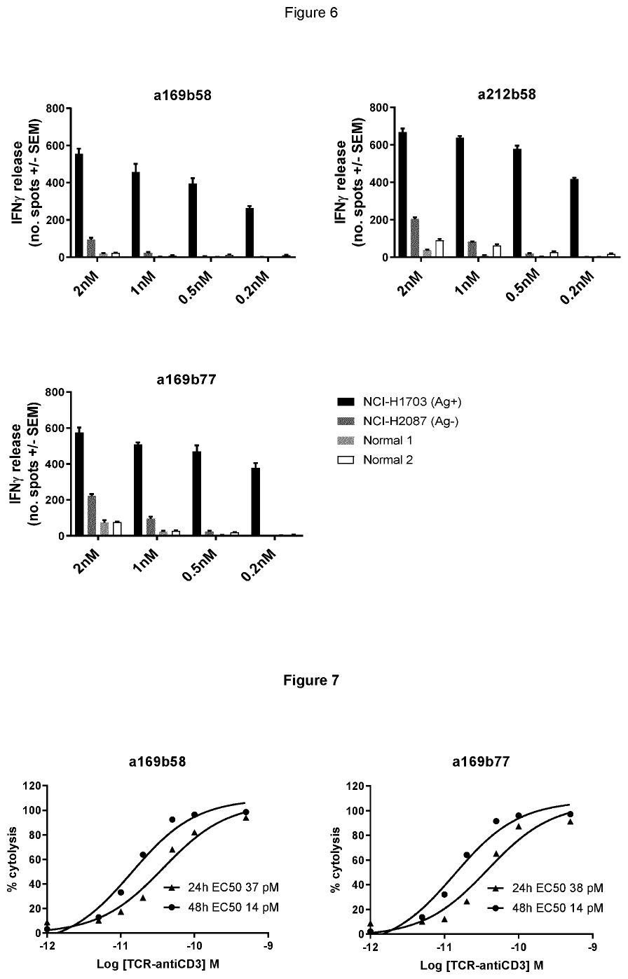

FIG. 6 shows that TCR-antiCD3 fusion protein of the invention give only minimal activation of T cells against normal cells derived from two different tissues within a therapeutically relevant range (≤1 nM).

### Example 4—Potent Killing of Tumour Cells

The ability of TCR-antiCD3 fusion molecules to mediate potent T cell mediated killing of antigen positive tumour cells was investigated using the xCELLigence assay (Acea Biosciences) according to the manufacturer's instructions. Briefly, 50 μl R10 was added to all wells in the xCELLigence E-plates left to equilibrate for ˜1 hour and a background reading taken. Target cells were counted and plated in 50 μl at final concentrations between 500 pM and 5 nM. The plate was left to equilibrate for 30 minutes prior to placing in the incubator. Sweeps were set for every 30 mins. Effectors (PBMCs) were taken out and left overnight at 37° C. in a flask to remove monocytes. The next day 50 μl R10 was removed from wells where peptide will be added. 50 μl ImmTAC (4× dilution) and 50 ul effectors were added to give 5:1 E:T ratio. 50 μl peptide (4× dilution) was added where necessary to give 10 μM peptide final concentration and 1 nM ImmTAC. The volume in each well was made up to 200 ul with R10 and sweeps were set for every 2 hours for 198 hours (100 sweeps). Percentage cytolysis was calculated at each concentration after 24 h and 48 h, and the response curves plotted in PRISM.

### Results

FIG. 7 shows response curves generated using TCR-antiCD3 fusion proteins of the invention. In each case Ec50 values were in the low pM range (<50 pM) at each time point, demonstrating potent killing of antigen positive cancer cells.

1. A specific binding molecule having the property of binding to KVLEYVIKV (SEQ ID NO: 1) HLA-A*02 complex and/or KVLEYVIKV (SEQ ID NO: 1) HLA-A*02 complex and comprising a TCR alpha chain variable domain and/or a TCR beta chain variable domain each of which comprises FR1-CDR1-FR2-CDR2-FR3-CDR3-FR4 where FR is a framework region and CDR is a complementarity determining region, wherein
the alpha chain CDRs have the following sequences:
CDR1-
(SEQ ID NO: 15)
SSVPPY
CDR2-
(SEQ ID NO: 16)
YTSAATLV
CDR3-
(SEQ ID NO: 17)
AARPSSSNTGKLI
optionally with one or more mutations therein,
and/or
(b) the beta chain CDRs have the following sequences:
CDR1-
(SEQ ID NO: 18)
PRHDT
CDR2-
(SEQ ID NO: 19)
FYEKMQ
CDR3-
(SEQ ID NO: 20)
ASSFTGFDEQF
optionally with one or more mutations therein

2. The specific binding molecule of claim 1, wherein the alpha chain variable domain framework regions comprise the following sequences:
FR1—amino acids 1-26 of SEQ ID NO: 2
FR2—amino acids 33-49 of SEQ ID NO: 2
FR3—amino acids 58-91 of SEQ ID NO: 2
FR4—amino acids 105-115 of SEQ ID NO: 2
or respective sequences having at least 90, at least 91, at least 92, at least 93, at least 94, at least 95, at least 96, at least 97, at least 98 or at least 99% identity to said sequences, and/or the beta chain variable domain framework regions may comprise the following sequences:
FR1—amino acids 1-26 of SEQ ID NO: 3
FR2—amino acids 32-48 of SEQ ID NO: 3
FR3—amino acids 55-91 of SEQ ID NO: 3
FR4—amino acids 103-112 of SEQ ID NO: 3
or respective sequences having at least 90, at least 91, at least 92, at least 93, at least 94, at least 95, at least 96, at least 97, at least 98 or at least 99% identity to said sequences.

3. The specific binding molecule of claim 1, wherein the one or more of the mutations in the alpha chain CDRs are selected from (with reference to the numbering of SEQ ID NO: 2): insertion of amino acids ARWG (SEQ ID NO: 43) after position 26, S27D, S28G, S52G, A53G, A54D, T55L, 156V, S97D, and S98A.

4. The specific binding molecule of claim 3, wherein
the alpha chain CDR1 comprises the following sequence: SSVPPY (SEQ ID NO: 15) or ARWGDGVPPY (SEQ ID NO: 21);
the alpha chain CDR2 comprises the following sequence: YTSAATLV (SEQ ID NO: 16) or
YTGGDLVV (SEQ ID NO: 22); and/or
the alpha chain CDR3 comprises the following sequence: AARPSSSNTGKLI (SEQ ID NO: 17), AARPSDSNTGKLI (SEQ ID NO: 23) or AARPSSANTGKLI (SEQ ID NO: 24).

5. The specific binding molecule of claim 1, wherein the one or more of the mutations in the beta chain CDRs are selected from (with reference to the numbering of SEQ ID NO: 3): Y50F, K52T, M53K, Q54F, F95V, T96W, G97D, F98W, and F98Y.

6. The specific binding molecule of claim 5, wherein
the beta chain CDR1 comprises the following sequence: PRHDT (SEQ ID NO: 18);
the beta chain CDR2 comprises the following sequence: FYEKMQ (SEQ ID NO: 19), FFETMF (SEQ ID NO: 25) or FFETKF (SEQ ID NO: 26); and/or
the beta chain CDR3 comprises the following sequence: ASSFTGFDEQF (SEQ ID NO: 20), ASSVWDWDEQF (SEQ ID NO: 27) or ASSVWDYDEQF (SEQ ID NO: 28).

7. The specific binding molecule of claim 1, which has one of the following combinations of alpha and beta CDRs:

| | Alpha | | | Beta | | |
|---|---|---|---|---|---|---|
| | CDR1 | CDR2 | CDR3 | CDR1 | CDR2 | CDR3 |
| a | ARWGDGVPPY | YTGGDLVV | AARPSSSNTGKLI | PRHDT | FFETMF | ASSVWDWDEQF |
| | (SEQ ID | (SEQ ID | SEQ ID NO: | (SEQ ID | (SEQ ID | (SEQ ID NO: 27) |
| | NO: 21) | NO: 22) | 17) | NO: 18) | NO: 25) | |
| b | ARWGDGVPPY | YTGGDLVV | AARPSSSNTGKLI | PRHDT | FFETKF | ASSVWDYDEQF |
| | (SEQ ID | (SEQ ID | (SEQ ID NO: | (SEQ ID | (SEQ ID | (SEQ ID NO: 28) |
| | NO: 21) | NO: 22) | 17) | NO: 18) | NO: 26) | |
| c | ARWGDGVPPY | YTGGDLVV | AARPSDSNTGKLI | PRHDT | FFETMF | ASSVWDWDEQF |
| | (SEQ ID | (SEQ ID | (SEQ ID NO: | (SEQ ID | (SEQ ID | (SEQ ID NO: 27) |
| | NO: 21) | NO: 22) | 23) | NO: 18) | NO: 25) | |
| d | ARWGDGVPPY | YTGGDLVV | AARPSDSNTGKLI | PRHDT | FFETMF | ASSVWDYDEQF |
| | (SEQ ID | (SEQ ID | (SEQ ID NO: | (SEQ ID | (SEQ ID | (SEQ ID NO: 28) |
| | NO: 21) | NO: 22) | 23) | NO: 18) | NO: 25) | |
| e | ARWGDGVPPY | YTGGDLVV | AARPSSANTGKLI | PRHDT | FFETMF | ASSVWDYDEQF |
| | (SEQ ID | (SEQ ID | (SEQ ID NO: | (SEQ ID | (SEQ ID | (SEQ ID NO: 28) |
| | NO: 21) | NO: 22) | 24) | NO: 18) | NO: 25) | |

8. The specific binding molecule of claim 1, wherein the alpha chain variable domain comprises any one of the amino acid sequences of SEQ ID NOs: 4-6 or a sequence having at least 90% identity thereto, and the beta chain variable domain comprises any one of the amino acid sequences of SEQ ID NOs: 7-9 or a sequence having at least 90% identity thereto.

9. The specific binding molecule of claim 1, wherein the alpha and beta chain variable domain pairs are selected from one of the following:
(a) the alpha chain variable domain of SEQ ID NO: 4 and the beta chain variable domain of SEQ ID NO: 7;
(b) the alpha chain variable domain of SEQ ID NO: 4 and the beta chain variable domain of SEQ ID NO: 8:
(c) the alpha chain variable domain of SEQ ID NO: 5 and the beta chain variable domain of SEQ ID NO: 7;
(d) the alpha chain variable domain of SEQ ID NO: 5 and the beta chain variable domain of SEQ ID NO: 9; and
(e) the alpha chain variable domain of SEQ ID NO: 6 and the beta chain variable domain of SEQ ID NO: 9.

10. The specific binding molecule of claim 1, wherein the binding molecule is an alpha-beta heterodimer further comprising an alpha chain TRAC constant domain sequence and a beta chain TRBC1 or TRBC2 constant domain sequence.

11. The specific binding molecule of claim 10, wherein the alpha and beta chain constant domain sequences are modified by truncation or substitution to delete a native disulfide bond between Cys4 of exon 2 of TRAC and Cys2 of exon 2 of TRBC1 or TRBC2.

12. The specific binding molecule of claim 10, wherein the alpha and/or beta chain constant domain sequence(s) are modified by substitution of cysteine residues for Thr 48 of TRAC and Ser 57 of TRBC1 or TRBC2, the said cysteines forming a non-native disulfide bond between the alpha and beta constant domains of the TCR.

13. The specific binding molecule of claim 1, wherein the specific binding molecule has a single chain format of the type Vα-L-Vβ, Vβ-L-Vα, Vα-Cα-L-Vβ, Vα-L-Vβ-Cβ, wherein Vα and Vβ are TCR α and β variable regions respectively, Cα and Cβ are TCR α and β constant regions respectively, and L is a linker sequence.

14. The specific binding molecule of claim 13, wherein the linker sequence is selected from the group consisting of GGGGS (SEQ ID No: 29), GGGSG (SEQ ID No: 30), GGSGG (SEQ ID No: 31), GSGGG (SEQ ID No: 32), GSGGGP (SEQ ID No: 33), GGEPS (SEQ ID No: 34), GGEGGGP (SEQ ID No: 35), GGEGGGSEGGGS (SEQ ID No: 36), GGGSGGGG (SEQ ID NO: 37). GGGS (SEQ ID No: 38), GGGGS (SEQ ID No: 39), TVLRT (SEQ ID No: 40), TVSSAS (SEQ ID No: 41) and TVLSSAS (SEQ ID No: 42).

15. The specific binding molecule of claim 1, further comprising a detectable label, a therapeutic agent and/or a PK modifying moiety.

16. The specific binding molecule of claim 15, wherein an anti-CD3 antibody is covalently linked to the C- or N-terminus of the alpha or beta chain of the specific binding molecule, optionally via a linker sequence.

17. A specific binding molecule-anti-CD3 fusion molecule, comprising an alpha chain variable domain which comprises:
an amino acid sequence selected from SEQ ID NOs: 4-6 or a sequence having at least 90% identity thereto,
a beta chain variable domain which comprises an amino acid sequence selected from SEQ ID NO: 7-9 or a sequence having at least 90% identity thereto, and
an anti-CD3 antibody, optionally comprising an amino acid sequence selected from SEQ NOS: 12-14, covalently linked to the N-terminus or C-terminus of the beta chain, optionally via a linker sequence.

18. The fusion molecule of claim 17, comprising:
an alpha variable domain having the sequence of SEQ ID NO 4 or a sequence at least 90% identical thereto and an alpha chain constant domain having the sequence of SEQ ID No 10 or a sequence at least 90% identical thereto, a beta variable domain having the sequence of SEQ ID NO 8 or a sequence at least 90% identical thereto and a beta chain constant domain having the sequence of SEQ ID No 11 or a sequence at least 90% identical thereto; and an anti-CD3 scFv antibody fragment having the sequence of SEQ ID NO 12 or a sequence at least 90% identical thereto fused to the N terminus of the beta chain via a linker having the sequence of SEQ ID NO: 29 or a sequence at least 90% identical thereto, or
an alpha variable domain having the sequence of SEQ ID NO 4 or a sequence at least 90% identical thereto and an alpha chain constant domain having the sequence of SEQ ID No 10 or a sequence at least 90% identical thereto, a beta variable domain having the sequence of SEQ ID NO 8 or a sequence at least 90% identical thereto, and a beta chain constant domain having the sequence of SEQ ID No 11 or a sequence at least 90% identical thereto; and an anti-CD3 scFv antibody fragment having the sequence of SEQ ID NO 14 or a sequence at least 90% identical thereto fused to the N terminus of the beta chain via a linker having the sequence of SEQ ID NO: 29 or a sequence at least 90% identical thereto.

19. A nucleic acid encoding an alpha chain and/or a beta chain as claimed in claim 1.

20. An expression vector comprising the nucleic acid of claim 19.

21. A cell harboring
(a) an expression vector encoding alpha and beta variable chains as claimed in claim 1, in a single open reading frame, or two distinct open reading frames; or
(b) a first expression vector which comprises nucleic acid encoding the alpha variable chain of a specific binding molecule as claimed in claim 1, and a second expression vector which comprises nucleic acid encoding the beta variable chain of a specific binding molecule as claimed in claim 1.

22. A non-naturally occurring and/or purified and/or engineered cell, especially a T-cell, presenting a specific binding molecule as claimed in claim 1.

23. A pharmaceutical composition comprising a specific binding molecule as claimed in claim 1, together with one or more pharmaceutically acceptable carriers or excipients.

24.-25. (canceled)

26. A method of treating a human subject having cancer or a tumor, comprising administering to said subject in need thereof a pharmaceutically effective dose of a pharmaceutical composition according to claim 23.

27. A method of producing a specific binding molecule as claimed in claim 1, comprising a) maintaining a cell according to claim 21 or 22 under optimal conditions for expression of the specific binding molecule chains and b) isolating the specific binding molecule chains.


SEQUENCE LISTING (44 sequences)

SEQ ID NO: 1 - protein, 9 aa, Homo sapiens
KVLEYVIKV

SEQ ID NO: 2 - protein, 115 aa, Homo sapiens
AQSVTQLGSHVSVSEGALVLLRCNYSSSVPPYLFWYVQYPNQGLQLLLKYTSAATLVKGINGFEAEFKKSETSFHLTKPSAHMSDAAEYFCAARPSSSNTGKLIFGQGTTLQVKP

SEQ ID NO: 3 - protein, 112 aa, Homo sapiens
AAGVIQSPRHLIKEKRETATLKCYPIPRHDTVYWYQQGPGQDPQFLISFYEKMQSDKGSIPDRFSAQQFSDYHSELNMSSLELGDSALYFCASSFTGFDEQFFGPGTRLTVL

SEQ ID NO: 4 - protein, 119 aa, Artificial Sequence
  Description of Artificial Sequence Synthetic polypeptide
  mutant
AQSVTQLGSHVSVSEGALVLLRCNYSARWGDGVPPYLFWYVQYPNQGLQLLLKYTGGDLVVKGINGFEAEFKKSETSFHLTKPSAHMSDAAEYFCAARPSSSNTGKLIFGQGTTLQVKP

SEQ ID NO: 5 - protein, 119 aa, Artificial Sequence
  Description of Artificial Sequence Synthetic polypeptide
  mutant
AQSVTQLGSHVSVSEGALVLLRCNYSARWGDGVPPYLFWYVQYPNQGLQLLLKYTGGDLVVKGINGFEAEFKKSETSFHLTKPSAHMSDAAEYFCAARPSDSNTGKLIFGQGTTLQVKP

SEQ ID NO: 6 - protein, 119 aa, Artificial Sequence
  Description of Artificial Sequence Synthetic polypeptide
  mutant
AQSVTQLGSHVSVSEGALVLLRCNYSARWGDGVPPYLFWYVQYPNQGLQLLLKYTGGDLVVKGINGFEAEFKKSETSFHLTKPSAHMSDAAEYFCAARPSSANTGKLIFGQGTTLQVKP

SEQ ID NO: 7 - protein, 112 aa, Artificial Sequence
  Description of Artificial Sequence Synthetic polypeptide
  mutant
AAGVIQSPRHLIKEKRETATLKCYPIPRHDTVYWYQQGPGQDPQFLISFFETMFSDKGSIPDRFSAQQFSDYHSELNMSSLELGDSALYFCASSVWDWDEQFFGPGTRLTVL

SEQ ID NO: 8 - protein, 112 aa, Artificial Sequence
  Description of Artificial Sequence Synthetic polypeptide
  mutant
AAGVIQSPRHLIKEKRETATLKCYPIPRHDTVYWYQQGPGQDPQFLISFFETKFSDKGSIPDRFSAQQFSDYHSELNMSSLELGDSALYFCASSVWDYDEQFFGPGTRLTVL

SEQ ID NO: 9 - protein, 112 aa, Artificial Sequence
  Description of Artificial Sequence Synthetic polypeptide
  mutant
AAGVIQSPRHLIKEKRETATLKCYPIPRHDTVYWYQQGPGQDPQFLISFFETMFSDKGSIPDRFSAQQFSDYHSELNMSSLELGDSALYFCASSVWDYDEQFFGPGTRLTVL

SEQ ID NO: 10 - protein, 86 aa, Homo sapiens
DIQNPDPAVYQLRDSKSSDKSVCLFTDFDSQTNVSQSKDSDVYITDKCVLDMRSMDFKSNSAVAWSNKSDFACANAFNNSIIPEDT

SEQ ID NO: 11 - protein, 130 aa, Homo sapiens
EDLNKVFPPEVAVFEPSEAEISHTQKATLVCLATGFYPDHVELSWWVNGKEVHSGVCTDPQPLKEQPALNDSRYALSSRLRVSATFWQDPRNHFRCQVQFYGLSENDEWTQDRAKPVTQIVSAEAWGRAD

SEQ ID NO: 12 - protein, 253 aa, Homo sapiens
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYTMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 13 - protein, 253 aa, Homo sapiens
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTISVDKSKNTAYLQMNSLRAEDTAVYYCARSGYYGDSDWYFDVWGQGTLVTVSS

SEQ ID NO: 14 - protein, 253 aa, Homo sapiens
AIQMTQSPSSLSASVGDRVTITCRASQDIRNYLNWYQQKPGKAPKLLIYYTSRLESGVPSRFSGSGSGTDYTLTISSLQPEDFATYYCQQGNTLPWTFGQGTKVEIKGGGGSGGGGSGGGGSGGGGSGGGSEVQLVESGGGLVQPGGSLRLSCAASGYSFTGYAMNWVRQAPGKGLEWVALINPYKGVSTYNQKFKDRFTFSVDKSKNTAYLQMNSLRAEDTAVYYCARSGYYGDSDWYFDVWGQGTLVTVSS

SEQ ID NO: 15 - protein, 6 aa, Homo sapiens
SSVPPY

SEQ ID NO: 16 - protein, 8 aa, Homo sapiens
YTSAATLV

SEQ ID NO: 17 - protein, 13 aa, Homo sapiens
AARPSSSNTGKLI

SEQ ID NO: 18 - protein, 5 aa, Homo sapiens
PRHDT

SEQ ID NO: 19 - protein, 6 aa, Homo sapiens
FYEKMQ

SEQ ID NO: 20 - protein, 11 aa, Homo sapiens
ASSFTGFDEQF

SEQ ID NO: 21 - protein, 10 aa, Homo sapiens
ARWGDGVPPY

SEQ ID NO: 22 - protein, 8 aa, Artificial Sequence
  Description of Artificial Sequence Synthetic peptide
  mutant
YTGGDLVV

SEQ ID NO: 23 - protein, 13 aa, Artificial Sequence
  Description of Artificial Sequence Synthetic peptide
  mutant
AARPSDSNTGKLI

SEQ ID NO: 24 - protein, 13 aa, Artificial Sequence
  Description of Artificial Sequence Synthetic peptide
  mutant
AARPSSANTGKLI

SEQ ID NO: 25 - protein, 6 aa, Artificial Sequence
  Description of Artificial Sequence Synthetic peptide
  mutant
FFETMF

SEQ ID NO: 26 - protein, 6 aa, Artificial Sequence
  Description of Artificial Sequence Synthetic peptide
  mutant
FFETKF

SEQ ID NO: 27 - protein, 11 aa, Artificial Sequence
  Description of Artificial Sequence Synthetic peptide
  mutant
ASSVWDWDEQF

SEQ ID NO: 28 - protein, 11 aa, Artificial Sequence
  Description of Artificial Sequence Synthetic peptide
  mutant
ASSVWDYDEQF

SEQ ID NO: 29 - protein, 5 aa, Homo sapiens
GGGGS

SEQ ID NO: 30 - protein, 5 aa, Homo sapiens
GGGSG

SEQ ID NO: 31 - protein, 5 aa, Homo sapiens
GGSGG

SEQ ID NO: 32 - protein, 5 aa, Homo sapiens
GSGGG

SEQ ID NO: 33 - protein, 6 aa, Homo sapiens
GSGGGP

SEQ ID NO: 34 - protein, 5 aa, Homo sapiens
GGEPS

SEQ ID NO: 35 - protein, 7 aa, Homo sapiens
GGEGGGP

SEQ ID NO: 36 - protein, 12 aa, Homo sapiens
GGEGGGSEGGGS

SEQ ID NO: 37 - protein, 8 aa, Homo sapiens
GGGSGGGG

SEQ ID NO: 38 - protein, 4 aa, Homo sapiens
GGGS

SEQ ID NO: 39 - protein, 5 aa, Homo sapiens
GGGGS

SEQ ID NO: 40 - protein, 5 aa, Homo sapiens
TVLRT

SEQ ID NO: 41 - protein, 6 aa, Homo sapiens
TVSSAS

SEQ ID NO: 42 - protein, 7 aa, Homo sapiens
TVLSSAS

SEQ ID NO: 43 - protein, 4 aa, Artificial Sequence
  Description of Artificial Sequence Synthetic peptide
ARWG

SEQ ID NO: 44 - protein, 20 aa, Artificial Sequence
  Description of Artificial Sequence Synthetic peptide
QRLMEDICLPRWGCLWEDDF